(19) United States
(12) Patent Application Publication
(10) Pub. No.: US 2006/0241978 A1
(43) Pub. Date: Oct. 26, 2006

(54) Electronic clinical chart system

(75) Inventor: Hiroto Yoshii, Shinagawa-ku (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Ohta-ku (JP)

(21) Appl. No.: 11/407,248

(22) Filed: Apr. 20, 2006

(30) Foreign Application Priority Data
Apr. 22, 2005 (JP) 2005-125252

(51) Int. Cl. G06F 19/00; G01N 33/48

(52) U.S. Cl. 705/3; 702/20

(57) ABSTRACT

An electronic clinical chart system having a search function based on comparison including measurement data obtained at the examination stage when a doctor in charge searches for similar case patients showing a symptom and the results of examinations similar to those of a new patient from past cases stored in the electronic clinical chart when making a diagnosis for a disease cause of the new patient is provided. The electronic clinical chart system is an electronic clinical chart system including: a patient electronic clinical chart database storing patients' examination data, clinical finding data and treatment data as digitized clinical chart information of patients; an input device inputting examination data, clinical finding data and treatment data as clinical chart information of a new patient; an extraction device extracting clinical chart information of similar case patients similar to the input clinical chart information of the new patient from the database; and a clinical chart information disclosing device disclosing to a viewer the contents of the extracted clinical chart information of similar case patients.

### BACKGROUND OF THE INVENTION

1. Field of the Invention

The present invention relates to an electronic clinical chart system comprising a patient database electronically storing clinical data (treatment data and examination data) and the like of patients and a search process device searching desired data from the patient database. The present invention relates particularly to an electronic clinical chart system comprising a search process device searching past patients' clinical data including similar examination data and the like in contrast with new patients' examination data from an existing patient database.

2. Related Background Art

In a medical institution, patients' profile, medical history, clinical finding and examination data and health care information such as medical treatment records are collected, recorded and managed as clinical charts. Previously, the clinical chart was prepared on a paper basis, but has been digitized using an electronic filing system. A certain form has been established regarding items to be described and a form for description of the items in digitalization of the clinical chart. For example, in consultation by a doctor, a profile of a patient, details leading to the onset, and information of a subjective symptom which are obtained by an interview, and information of an objective symptom which is found in consultation by the doctor are each categorized into predetermined items, and then recorded on an electronic clinical chart as a part of clinical finding data using specific formalized expressions. When identifying a disease cause of the patient's symptom, an inference is made using diagnostic knowledge based on information of the subjective symptom and information of objective symptom, and probable disease cause candidates are selected. Further, various examinations are carried out for identifying a real disease cause of the probable disease cause candidates. The results of examinations carried out are each categorized into predetermined items, and then recorded on the electronic clinical chart as examination data using specific formalized expressions. Finally, using a profile of a patient, details leading to the onset, information of a subjective symptom, information of an objective symptom and the results of various examinations, the most probable disease cause of probable disease cause candidates inferred beforehand is identified and a diagnosis is made. In consideration of the made diagnosis (disease cause), treatment means which is considered reasonable is selected from a plurality of treatment means, and treatments are started.

Thereafter, a series of medical treatment records including treatments carried out during a medical treatment period and changes in the symptom of the patient are recorded on the electronic clinical chart as treatment data.

In the field of electronic clinical chart, a system supporting a task of making an inference using diagnostic knowledge and selecting probable disease cause candidates when a doctor identifies a disease cause, making use of an advantage that a profile of a patient, details leading to the onset, information of a subjective symptom and information of an objective symptom constituting a part of clinical finding data, or the results of various examinations stored in examination data are stored in a predetermined format has been proposed (see Japanese Patent Application Laid-Open No. H10-177605). In this case, most of abnormal examination data associated with diseases included in the results of examinations match most probable disease cause candidates, but there have been cases where some abnormal examination data do not match the most probable disease cause. If such mismatch occurs, it is necessary to review probable disease cause candidates and then identify the most probable disease cause candidate matching all of abnormal examination data associated with the disease. The electronic clinical chart system of the aforementioned document has a function of supporting a task making an inference using diagnostic knowledge including information of the subjective symptom of the patient, information of the objective symptom and the results of examinations, and reviewing and reselecting probable disease cause candidates. That is, the diagnosis support function employed by the electronic clinical chart system of the aforementioned document has an effect of alleviating the fear of missing the “real disease cause” of a patient which occurs when a doctor misses mismatch or fails to give sufficient consideration when some abnormal examination data do not match the most probable disease cause candidate.

The conventional electronic clinical chart system stores information of the subjective symptom of the patient, information of the objective symptom, and results of examinations as clinical chart information of each patient. However, the results of examinations stored are only results of examinations for which it is not necessary to further make a determination as to medical substances implicated by examination data based on numerical information obtained in examinations, such as, for example, a blood cholesterol concentration, a blood glucose level and an urine acid level. That is, whether or not abnormal numerical data exists in the results of examinations of the patient is determined according to already established determination criteria with reference to the results of examinations of normal healthy persons. Thus, although it has a function of supporting a doctor's task of reviewing and reselecting probable disease cause candidate, abnormal examination data are extracted only in case that it can be interefered with high accuracy based on a typical cases. That is, the system covers examination items for which inspection criteria on whether normal or abnormal, which are used for extraction of abnormal examination data included in the results of examinations, have already been established. Only in this case, the diagnosis support function of the electronic clinical chart system described above is effective.

### SUMMARY OF THE INVENTION

In addition to a symptom of a patient and the results of examinations which have been used, examination data obtained in examination belonging to a leading edge medical field, such as, for example, gene examinations is also used when a doctor identifies a disease cause of the patient. In the examinations belonging to a leading edge medical field, however, there are not a few cases where it is difficult to clearly interpret what is implicated by obtained examination data based on the examination data. For example, for patients with infectious diseases, a probe hybridization method using a DNA microarray comprising DNA probes for detection of a plurality of species of bacteria for identification of causal microorganism of the infectious disease is applied. In this case, there are not a few cases where even if causal microorganism belonging to the same species are included in plural of samples, there is a difference in signal intensity of labels added to target nucleic acid molecules for detection, which are observed at spots of the probes on the DNA microarray. Therefore, there may be cases where it is not easy to interpret the results of examinations in samples taken from individual patients with high accuracy merely by referring to the results of examinations using a typical DNA microarray for causal microorganism belonging to the same species. Thus, it is desired to propose a method allowing a high-accuracy diagnosis to be performed using detection data when it is possible that examination data for use in the diagnosis shows a non-neglectable deviation as compared to typical examination data measured in presumed disease cause candidates.

The present invention solves such a problem, and it is an object of the present invention to provide an electronic clinical chart system having a function of providing medical information available in performing a high-accuracy diagnosis to a doctor taking charge of the diagnosis and treatment of a new patient to support the diagnosis using detection data if there is the possibility that examination data for use in the diagnosis shows a deviation that is not negligible as compared to typical examination data measured in presumed disease cause candidates.

The aforementioned problem is solved by constructing an electronic clinical chart system having the following configuration.

The present inventor has found that if there is the possibility that examination data for use in a diagnosis shows a deviation that is not negligible as compared to typical examination data measured in presumed disease cause candidates, information on whether or not any of examination data in patients with presumed disease causes show a deviation similar to that of examination data in a new patient by careful inspection of past cases is extremely useful. That is, if past cases showing a deviation similar to that of examination data in a new patient can be selected, those cases can be used as reference cases in presuming a factor causing examination data in the patient to show a deviation as compared to typical examination data measured in presumed disease cause candidates. Further, by conducting a diagnosis using the detection data referring to past cases similar to those cases as well, the accuracy of the resulting diagnosis is dramatically improved.

In the electronic clinical chart system according to the present invention, patients' examination data, clinical finding data and treatment data are stored in a patient electronic clinical chart database storing digitized clinical chart information of each patient for the purpose of carefully inspecting past cases including the aforementioned examination data. Then, a device comparing new patient's examination data and clinical finding data input as digitized clinical chart information from an input apparatus with examination data and clinical finding data of past cases (patients) recorded in the patient electronic clinical chart database, and selecting past cases (patients) showing the similar symptom and examination data is added. A device disclosing clinical chart information, i.e. examination data, clinical finding data and treatment data, of the selected past cases (patients) as reference cases to a viewer in a medical field involved in a treatment, e.g. a doctor taking charge of a new patient is provided.

That is, the electronic clinical chart system according to the present invention is an electronic clinical chart system comprising:

a patient electronic clinical chart database storing Patients' examination data, clinical finding data and treatment data as digitized clinical chart information of each patient;

an input device inputting examination data, clinical finding data and treatment data as clinical chart information of a new patient;

an extraction device extracting clinical chart information of similar case patients which is similar to the input clinical chat information of the new patient, from the database; and

a clinical chart information disclosing device disclosing the contents of the extracted clinical chart information of similar case patients to a viewer.

In the electronic clinical chart system according to the present invention, patients' examination data, clinical finding data and treatment data are stored in the patient electronic clinical chart database, and the contents of the electronic clinical chart database including detailed examination data can carefully be inspected to extract similar past cases (patients) showing a high similarity to examination data and clinical finding data of a new patient. Further, by disclosing examination data, clinical finding data and treatment data of the selected similar past cases (patients) to a doctor in charge as reference cases when diagnosing a new patient, a more useful diagnosis support function is provided.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a view schematically showing a general configuration of an electronic clinical chart system according to the present invention;

FIG. 2 is a block diagram schematically showing a configuration of an information processing apparatus that is used for achievement of a function of searching similar cases in the electronic clinical chart system according to the present invention;

FIG. 3 is a view schematically showing a configuration of a system constructed as a client server type for the information processing apparatus that is used for achievement of a function of searching similar cases in the electronic clinical chart system according to the present invention;

FIG. 4 is a view schematically showing a flow of a step of detecting nucleic acid molecules contained in a sample using a DNA microarray;

FIG. 5 is a view schematically showing a procedure of a method for identifying causal microorganism of an infectious disease using DNA probes fixed on the DNA microarray, and a principle thereof; and

FIG. 6 is a view showing one example of a two-dimensional image representing a fluorescence intensity at each spot on the DNA microarray when causal microorganism of the infectious disease are identified as Staphylococcus aureus or Escherichia coli in the electronic clinical chart system according to the present invention.

### DESCRIPTION OF THE PREFERRED EMBODIMENTS

Preferred embodiments of the present invention will now be described in detail in accordance with the accompanying drawings.

An electronic clinical chart system according to the present invention is an electronic clinical chart system comprising:

a patient electronic clinical chart database storing patients' examination data, clinical finding data and treatment data as digitized clinical chart information of each patient;

an input device inputting examination data, clinical finding data and treatment data as clinical chart information of a new patient;

a similar case patient extracting device comparing examination data and clinical finding data of the new patient input as clinical chart information with patients' examination data and clinical finding data in past, cases stored in the patient electronic clinical chart database to select one or more of past case patients having examination data and clinical finding data similar to the examination data and clinical finding data of the new patient; and

a similar case patient clinical chart information disclosing device electronically disclosing in a viewable form the contents of clinical chart information of similar case patients selected by the similar case patient extracting device to a viewer, particularly to a doctor taking charge of the diagnosis and treatment of the new patient,

and its preferable forms may include the following aspect.

First, in the electronic clinical chart system according to the present invention, the similar case patient extracting device preferably has a function of ranking according to the level of similarity using the level of similarity to the examination data and clinical finding data of the new patient as an indicator for one or more of the selected similar case patients. Particularly, the function of ranking according to the level of similarity is preferably applied to select one of past case patients having examination data and clinical finding data most similar to the examination data and clinical finding data of the new patient in the similar case patient extracting device.

The similar case patient clinical chart information disclosing device preferably has a function of carrying out processing of making it impossible to view personal information which is not used in the diagnosis and treatment of similar case patients included in disclosed clinical chart information of the similar case patients, and is used only in identification of patients.

The patient electronic clinical chart database is preferably configured to normally include the details of conducted examinations and the results of measurements as a part of a database of the results of examinations and measurements included in patients' examination data constituting the database.

In this case, the details of conducted examinations and the results of measurements included as a part of the database of the results of examinations and measurements included in patients' examination data may include digitized data showing the results of probe hybridization reactions for a plurality of DNA probes using a DNA microarray.

For example, digitized data showing the results of probe hybridization reactions for a plurality of DNA probes using the DNA microarray may include scan image data of label-originated signal intensities including spot regions of a plurality of DNA probes or label-originated signal intensity data at spots of a plurality of DNA probes when the results of probe hybridization reactions for a plurality of DNA probes are shown by label-originated signal intensities for identifying causal microorganism of an infectious disease.

Particularly, the details of conducted examinations and the results of measurements included as a part of the database of the results of examinations and measurements included in patients' examination data preferably include digitized data showing the results of probe hybridization reactions for a plurality of DNA probes for identifying causal microorganism of an infectious disease using the DNA microarray.

Preferred embodiments of the present invention will be described below in accordance with the drawings.

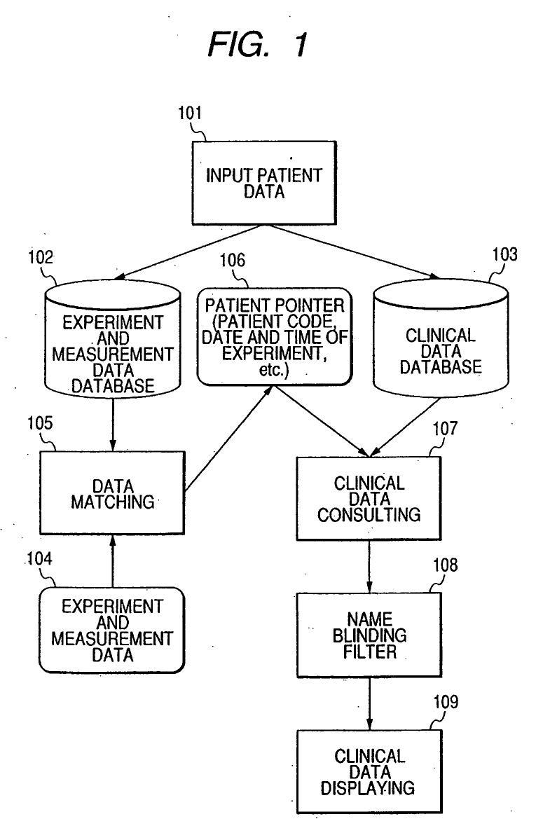

FIG. 1 is a view for explaining the concept of processing procedures in a device selecting similar case patients and a similar case patient clinical chart information disclosing device, which are employed in the electronic clinical chart system according to the present invention.

In the electronic clinical chart system according to the present invention, patients' examination data, clinical finding data and treatment data are stored on a patient electronic clinical chart database as clinical chart information of patients in accordance with a predetermined formalized form. To the clinical chart information of patients are added indexes (patient codes) showing that the information is information about the patients. Since information is added in time sequence after the diagnosis of the patient is started until the treatment is completed, time indexes showing a time when the information was recorded are also added. For example, in various kinds of examination data, the date and time when a sample for use in a certain examination was taken from a patient is significant, and therefore in addition to a time when the examination result was recorded, a date and time when the sample was taken is recorded together with the examination result. Further, examination data includes the results of examinations that are repeatedly conducted for the purpose of observing the course of the symptom of the patient, for example basic examination data such as body temperature, and the results of examinations that are sporadically conducted for the purpose of use in diagnoses, for example examination data of blood examinations, infectious bacteria examinations and the like. The basic examination data is data that is accumulated in time sequence, and is recorded separately from specific examination data as the results of examinations that are sporadically conducted. For the results of examinations that are sporadically conducted as specific examination data, data corresponding to raw data obtained in actual examinations is also recorded, and the doctor can view not only determination results (secondary information) by persons in charge of examination based on examination results extracted from raw data, but also raw data recorded as electronic image information and examination results (primary information) extracted from the raw data as required.

On clinical finding data are recorded information of a subjective symptom of a patient, a course until the onset of the symptom, a treatment conducted after the onset of the symptom and until consultation, and the like, which is obtained through interviews by a doctor at the time of consultation, information of an objective symptom and the like observed by consultation of the patient by the doctor, information obtained during consultation prior to the treatment, and information of an original diagnosis result, disease name and the like determined by the doctor referring to various kinds of examination results. The diagnosis result and disease name may be corrected to a more proper diagnosis result and disease name in the process of the treatment, and in this case, the corrected item is added to diagnosis finding data one by one together with the ground for the correction. At the time of completion of the full treatment, a disease cause causing the symptom of the patient and a relationship between the disease cause and the symptom shown by the patient are finally recorded on clinical finding data to create past case information. In addition, the profile of the patient, e.g. records of the name, age, gender and previous disease of the patient, information about a health condition other than the condition of the disease, and the like constitute a part of clinical finding data.

For the treatment data, information of medical treatments determined and conducted by a doctor in charge is recorded in time sequence. At the same time, information about a course of the symptom of the patient is recorded in time sequence. The treatment conducted for the patient and evaluations on the effectiveness of the treatment also constitute a part of treatment data.

In the electronic clinical chart system according to the present invention, patients' examination data, clinical finding data and treatment data are each input to the patient electronic clinical chart database as predetermined formalized information in a patient data inputting step 101. In the configuration shown in FIG. 1, the patient electronic clinical chart database is sectioned into two databases: an experiment and measurement data database 102 storing specific examination data as the results of examinations conducted sporadically; and a clinical data database 103 storing clinical finding data and treatment data, to which information is added in time sequence after the diagnosis of the patient is started until the treatment is completed.

In the conventional electronic clinical chart system, the patient electronic clinical chart database corresponds to the clinical data database 103 storing clinical finding data and treatment data. For the results of examinations conducted sporadically, the results of determination by persons in charge of examination are attached to electronic clinical charts of patients, but the raw data is obtained separately when it is necessary to actually examine raw data.

In the electronic clinical chart system according to the present invention, when past cases recorded in the patient electronic clinical chart database are contrasted with the symptom and the results of examinations of a new patient, information from sporadic examinations stored in the experiment and measurement data database 102 can be contrasted at the level of not only secondary information but also primary information, in addition to the outline of examination data and clinical finding data recorded in the clinical data database 103.

For example, in the case of the results of examinations by probe hybridization reactions for various kinds of DNA probes using a DNA microarray, there are cases wherein fluorescence labels are added to detection target nucleic acid molecules, and the fluorescent intensity associated with hybridization is measured at the spot of each probe. For the results of measurement, for example, scan image data of the DNA microarray and fluorescent intensity data obtained by extracting fluorescent intensities at the spots from the scan image data are stored in the experiment and measurement data database 102 as primary information which is electronically recorded. Then, determination results of identifying detection target nucleic acid molecules based on fluorescent intensity data of the spots are recorded as secondary information. The examination data includes data which is not required to be secondary information as a result of further determination from measurement values of primary information, such as the blood glucose level and the uric acid level.

As a method for detecting nucleic acid molecules using the “DNA microarray”, a detection method having a configuration other than the configuration in which fluorescent labels are added to the above-mentioned detection target nucleic acid molecules and the probe hybridization method is employed as a detection system may also be used. For example, a configuration in which a “single-chain elongation reaction” is made to proceed using single strand DNA fixed on the “DNA microarray” as a primer and using detection target nucleic acid molecules as a mold, and at this time, a fluorescent label is introduced into a nucleic acid chain elongated to the 3′ side of single strand DNA as a primer may be used. In this configuration, only when in a test sample, a base sequence complementary to single strand DNA fixed on the “DNA microarray” is contained, and at the same time, detection target nucleic acid molecules forming a base pair with the 3′ terminal of single strand DNA exists, the nucleic acid chain is elongated by the “single-chain elongation reaction”. This characteristic can be used as a method for selectively detecting a nucleic acid molecule having a base complementary to the base of the 3′ terminal of single strand DNA when a plurality of kinds of nucleic acid molecules capable of forming hybrid bodies with single strand DNA exist.

The detection method using the “DNA microarray” can use a configuration of detecting a protein showing a specific binding ability for single strand DNA consisting of a specified base sequence, in addition to detecting a nucleic acid molecule having a base sequence complementary to single strand DNA. For example, there is a molecule called aptamer as a single strand nucleic acid molecule which is not a substrate of a protein itself but shows a specific binding ability for the protein. For example, the detection method can have a configuration in which a plurality of kinds of aptamer molecules consisting of single strand DNA are fixed on the “DNA microarray” to detect a plurality of kinds of target proteins for which the aptamer molecules show a specific binding ability.

For examinations conducted sporadically for the purpose of use in diagnoses and the like, examination items effective for identification of disease cause candidates that are presumed based on the symptom of the patient at the time when the doctor in charge examines the patient are selected. Conventionally, for the results of examinations for the selected examination items, whether the results of examinations of the patient are appropriate or not is determined on the basis of the results of examinations found in typical cases in presumed disease cause candidates, and the disease cause of the patient is identified from a plurality of presumed disease cause candidates.

There are not a few cases where when whether or not the results of examinations of the patient are within an appropriate range is determined on the basis of the results of examinations found in typical cases, there is a difference in several points as compared to the results of examinations found in typical cases, and it is not easy to determine whether or not the results are within an appropriate range. In this case, making a comparison with a past case of which the disease cause has been ultimately determined to be the concerned disease cause, and conducting a study on existence/nonexistence of a past case (patient) showing a difference as compared to the results of examinations found in typical cases is effective for improvement of the accuracy of diagnoses. That is, there are not a few cases where for patients with the same disease cause, the results of examinations deviate from the results of examinations found in typical cases due to the progress of the symptom or individual differences.

In the electronic clinical chart system according to the present invention, clinical finding data of a new patient which is input as clinical chart information as a diagnosis is conducted is data of the symptom of the patient and disease cause candidates presumed based on the symptom, which is input by a doctor in charge, and examination data is primary information of the results of examinations corresponding to raw data of examinations as the results of examinations for examination items effective for identification of the disease cause candidates, which is input by a department in charge of examination. In the similar case patient extracting device, examination data and clinical finding data of the new patient is directly compared with examination data and clinical finding data of patients in past cases stored in the patient electronic clinical chart database. In the comparison between clinical finding data of the new patient and clinical finding data of patients in past cases, the symptom of the new patient as primary information is contrasted with symptoms recorded in patients in past cases. In the comparison between examination data of the new patient and examination data of patients in past cases, primary information of the results of examinations corresponding to raw data of examinations is mutually contrasted.

For achieving a principal object of the present invention, a plurality of patients in past cases of which the recorded examination data is similar to examination data of the new patient are first selected. In this process, primary information of the results of examinations; experiment and measurement data 104 corresponding to examination data of the new patient, especially raw data of examinations input by a department in charge of examination is compared with experiment and measurement data of past cases (patients) stored in the experiment and measurement data database 102 in a data matching step 105. The experiment and measurement data of past cases (patients) is sorted in descending order of the level of similarity to the experiment and measurement data 104 of the new patient, and for the experiment and measurement data of a group of past cases (patients) in high order of sorting, indexes (patient pointers) of the past cases (patients) are listed. Then, in a similar patient clinical data consulting step 107, the clinical data database 103 is consulted based on patient pointers 106 of listed similar patients, and clinical finding data (symptom and established disease cause) of patients in past cases is captured as a data set for tasks. The patient pointer 106 refers to a series of index information of the patient code, the date and time of recording examination data is recorded, the date and time of experiment (examination), the date and time of taking examination samples, and the like for identifying clinical chart information of each case (patient).

A group of past cases (patients) selected as similar case patients are high rank cases (patients) sorted in descending order of the level of similarity, and the number of the past cases is appropriately determined. For example, if the number of cases of disease cause candidates presumed is extremely small, the number of past cases (patients) of a group selected as similar case patients is resultantly small. If the number of cases of disease cause candidates presumed is extremely large, the number of past cases (patients) of a group selected as similar case patients is large as a result of existence of a large number of cases in the same order having the same level of similarity. In the similar case patient clinical chart information disclosing device, clinical chart information of similar case patients selected by the similar case patient extracting device, especially clinical finding data and treatment data of similar case patients is electronically disclosed in a viewable form for a doctor involved in the diagnosis and treatment of the new patient. The disclosed contents include a disease cause causing the symptom of similar case patients, ultimately established at the time of completion of the full treatment, information about a relationship between the disease cause and the symptom shown by the patient, and information about a relationship between primary information of examination data and the ultimately established disease cause of similar case patients. The information is often very helpful in diagnosis of the disease cause of the new patient.

In clinical chart information of past cases, information corresponding to personal information of similar case patients is not required for comparison of cases, and is removed as confidential information from the data set for tasks. That is, a step 108 of removing personal information of similar patients by a name blinding filter is provided before a step 109 of displaying clinical data of similar patients from the data set for tasks created in the clinical data consulting step 107. In this process, only information passing through such a filter after separating confidential information by the name blinding filter is disclosed to a doctor involved in the diagnosis and treatment of a new patient as a viewer. At the same time, treatment data in similar case patients is disclosed and it is often helpful in determining a policy of treatment which is conducted on a new patient after diagnosis.

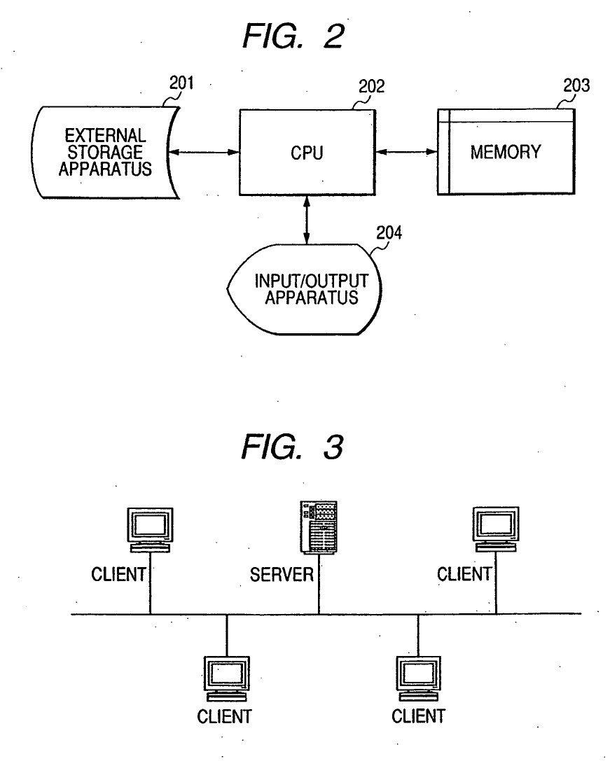

The similar case patient extracting device and similar case patient clinical chart information disclosing device provided in the electronic clinical chart system according to the present invention is effective when a diagnosis is conducted referring to examination data obtained by leading edge medical examinations such as, for example, gene examinations. For example, when an examination for identifying causal microorganism of an infectious disease is performed, fluorescent intensity data of probes of the DNA microarray that is measured may be different in some situation even though causal microorganism of the infectious disease are actually the same. There are not a few cases where it is not easy to make a univocal determination based on fluorescent intensity data of probes of the DNA microarray which is measured. If fluorescent intensity data of probes of the DNA microarray extremely similar for causal microorganism of the infectious disease ultimately established in past cases is measured, the accuracy of determination of examination data can dramatically be improved based on the comparison between examination data of the new patient and examination data of patients in past cases. FIG. 2 is a block diagram showing a basic configuration of an information processing apparatus available for information processing operations in the similar case patient extracting device and the similar case patient clinical chart information disclosing device provided in the electronic clinical chart system according to the present invention.

The electronic clinical chart system is constructed using an information processing apparatus consisting of an external storage apparatus 201, a central processing unit (CPU) 202, a memory 203 and an input/output apparatus 204. The external storage apparatus 201 is used for storage of the patient electronic clinical chart database, programs used in various kinds of information processing processes in the similar case patient extracting device and the similar case patient clinical chart information disclosing device, and the like. The central processing unit (CPU) 202 is used in processes of information processing and information management performed in the system. The memory 203 can be used for a purpose of temporarily recording programs, subroutines and data when performing processes of information processing and information management via the central processing unit (CPU) 202. The input/output apparatus 204 performs input operations for input of data that is recorded in the patient electronic clinical chart system database, control of parameters that are externally set for, for example, execution of programs when performing processes of information processing and information management, and so on, and various display and output operations. For example, when operating the similar case patient extracting device and the similar case patient clinical chart information disclosing device, interaction with a user (doctor) is performed via the input/output apparatus 204.

The electronic clinical chart system has a configuration in which a large number of users (doctors) use such a system in parallel, and at the same time, the patient electronic clinical chart database stored in the external storage apparatus is configured to be managed integrally on the overall system. Therefore, in many cases, a client server type system configuration shown in FIG. 3 is used. The patient electronic clinical chart database is stored in a server shared by a large number of clients. The patient electronic clinical chart database is sectioned into the experiment and measurement data database 102 and the clinical data database 103, and the sectioned databases are then stored in the server. A large number of clients are used by, for example, departments in charge of input and management of experiment and measurement data to experiment and measurement data database 102 and a group of doctors in charge of diagnosis and treatment of patients independently. When operating the similar case patient extracting device and the similar case patient clinical chart information disclosing device, persons who use the devices are limited to doctors, and the doctors interact with the system through the clients.

### Preferred Embodiments

A process of selecting a plurality of patients in past cases for which examination data similar to examination data of a new patient in the similar case patient extracting device and the similar case patient clinical chart information disclosing device provided in the electronic clinical chart system according to the present invention, especially in the similar case patient clinical chart information disclosing device will be described below with a specific example. Experiment and measurement data are mutually compared in the data matching step shown in FIG. 1, and examination data obtained by detecting detection target nucleic acid molecules contained in an examination sample using a DNA microarray will be taken as one example of the experiment and measurement data.

Not only for gene examinations using a DNA microarray, but also for gene examinations using, for example, a genome analysis technique such as the quantitative PCR, experiment and measurement data may be mutually compared in a similar form. Further, for a leading-edge medical examination technique in which it is very difficult to interpret experiment and measurement data, the effect of the present invention is exhibited because experiment and measurement data are mutually compared to select similar case patients from past cases without interpretation.

That is, the case that will be described below is one example of the most preferred embodiment according to the present invention, but the present invention is not limited to such an embodiment.

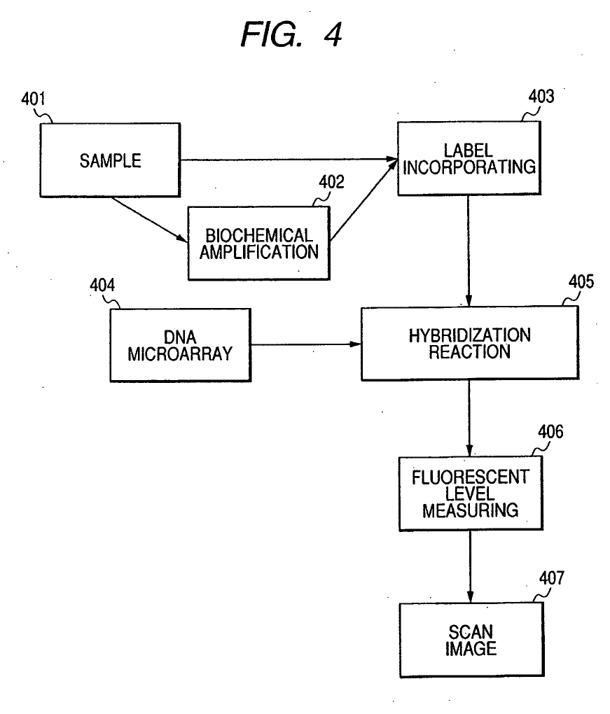

FIG. 4 is a view schematically showing a flow of a step of detecting nucleic acid molecules contained in a “sample” 401 by applying a probe hybridization method using a DNA microarray on which a plurality of kinds of DNA probes are fixed in the form of an array. The “sample” 401 is a liquid or solid sample presumed to contain detection target nucleic acid molecules. For example, when the patient is presumed to contract an infectious disease, the sample is a sample containing causal microorganism of the presumed infectious disease. In practice, among body fluids such as blood, sputum, gastric juice, vaginal discharges and intraoral mucosa, which can be taken from the patient, or excrements such as urine and feces, those expected to contain causal microorganism of the presumed infectious disease are selected as the sample 401.

Nucleic acid molecules originating from causal microorganism, which are used for identification of causal microorganism of the infectious disease presumed to be contained in the sample 401, are amplified in a “biochemical amplification” step 402 as necessary. For example, the base sequence of 16s rRNA varies depending on the species of individual bacteria, and bacteria can be identified by using the difference in the base sequence. When 16s rRNA having a specific base sequence for each species of bacteria is a detection target, a configuration in which genome DNA is extracted from bacteria, and then PCR amplification of only regions coding 16s rRNA is performed can be used. For most of causal microorganism of the infectious disease, the base sequences of regions coding 16s rRNA have been determined, and a primer for PCR amplification of the regions coding 16s rRNA is designed based on the known base sequence. A PCR amplification product corresponding to the regions coding 16s rRNA is prepared using as a mold the genome DNA extracted from bacteria and using the primer for PCR amplification. As a method for amplifying nucleic acid molecules, a method other than the PCR amplification method, such as a LAMP method, can be used in some cases.

When detecting detection target nucleic acid molecules using the probe hybridization method, it is desirable that the concentration of detection target nucleic acid molecules contained in a hybridization reaction solution should be set to a certain level or higher. Thus, a specimen DNA solution having a concentration of detection target nucleic acid molecules at a certain level or higher may also be prepared by using DNA fragments of a primary amplification product as a mold and further performing PCR amplification, as necessary.

For detection of hybridization of DNA probes and detection target nucleic acid molecules, detection target nucleic acid molecules itself are labeled, and the hybridization is detected via labels added to detection target nucleic acid molecules fixed on the DNA microarray with hybridization. Accordingly, labels are added to detection target nucleic acid molecules contained in the sample 401 or nucleic acid molecules of amplification products amplified in the biochemical amplification step 402 using various kinds of labeling methods in a “label incorporating” step 403. As a labeling material that is used for the labeling, a fluorescence labeling material for general purpose use, for example a fluorescent material such as Cy3, Cy5 or Rhodamine is preferably used. Alternatively, an intercalator type fluorescence labeling material may be added to nucleic acid molecules of amplification products via a linker. If these fluorescent labels are used, the amount of detection target nucleic acid molecules fixed with hybridization from spots of DNA probes fixed on the DNA microarray can be observed in the form of a two-dimensional image throughout the DNA microarray by the fluorescent intensity. Addition of labels using various kinds of labeling methods may be performed in the “label incorporating” step 403, or may be introduced when elongating the nucleic acid chain during the biochemical amplification step 402.

Using a DNA microarray chip prepared in a step 404 of designing a probe sequence and fixing DNA probes fabricated by chemical synthesis to form a DNA microarray in advance, the fixed DNA probes are made to undergo a reaction with target nucleic acid molecules labeled in the “label incorporating” step 403 in a hybridization reaction step 405.

In the step 404 of forming a DNA microarray, a plurality of kinds of DNA probes are spotted in the form of an array and fixed on the surface of a carrier for fixation of probes in accordance with a predetermined arrangement order. For the surface of the carrier for fixation of probes, a material to which target nucleic acid molecules are hard to nonselectively adsorb is selected. For example, a flat substrate such as a glass substrate, a plastic substrate or a silicon wafer is preferably used as the carrier (substrate) for fixation of probes. As long as a plurality of kinds of DNA probes can be spotted in the form of an array and fixed in accordance with a predetermined arrangement order, a three-dimensional structure having a rough surface, a spherical matter such as a bead, a bar, a string or a filament may be used aside from a flat substrate.

If a material to which target nucleic acid molecules are hard to nonselectively adsorb is selected for fixation of probes, for the surface of the carrier (substrate), it is difficult to densely fix DNA probes on the carrier (substrate) for fixation by adsorption. Thus, for fixation of DNA probes on the surface of the carrier (substrate) for fixation, a configuration in which they are coupled through covalent bonding is normally selected. Specifically, a configuration in which the surface of the carrier (substrate) for fixation is subjected to a surface treatment for additively introducing a functional group capable of being used for fixation of DNA probes onto the surface of the carrier (substrate) for fixation in advance, while a reactive functional group is introduced on the DNA probe side in advance, and the surface of the carrier for fixation and the DNA probes are coupled via covalent bonding by a reaction between the functional groups is preferably selected. When DNA probes and target nucleic acid molecules hybridize, labeled target nucleic acid molecules are quantitatively fixed at spots of the DNA probes as a result of stable fixation of DNA probes on the surface of the carrier (substrate) for fixation. Since in this state, the amount of labeled target nucleic acid molecules is detected using labels, the quantitative characteristic of detection is improved, leading to a preferable configuration.

As one example of means for stably fixing DNA probes onto the surface of the carrier (substrate) for fixation, a maleimide group is introduced onto the surface of the carrier (substrate) for fixation, while a thiol (—SH) group is introduced at the terminal of the DNA probe, and then fixation is achieved by covalent bonding through a reaction between the maleimide group and the thiol (—SH) group. For example, an amino silane coupling agent is made to react with the surface of a glass substrate to form fixing layer of the coupling agent. An EMCS reagent (N-(6-maleimidocaproyloxy) succinimide: manufactured by Dojin) is made to act on an amino group originating from the coupling agent to cause a reaction between the succinimide portion and the amino group, whereby a maleimide group originating from the EMCS reagent is introduced. For introduction of the SH group into single strand DNA, the thiol (—SH) group is introduced at the 5′ terminal of a DNA chain having a desired base sequence via a linker by using 5′-Thiol-Modifier C6 (manufactured by Glen Research) on a DNA automatic synthesizer when chemically synthesizing single strand DNA.

In addition, a configuration in which an epoxy group is introduced onto the surface of the carrier (substrate) for fixation, while an amino group is introduced at the 3′ terminal of single strand DNA, and a coupling is formed by a reaction between the amino group and the epoxy group cam also be used. For introduction of a functional group onto the surface of the carrier (substrate) for fixation, surface treatments with various kinds of silane coupling agents may suitably be used. A method in which using a functional group introduced by the silane coupling agent and a functional group introduced at the terminal of single strand DNA, the surface of the carrier and single strand DNA are coupled may be used. Further, a configuration in which a functional group is introduced onto the surface of the carrier (substrate) for fixation using a method of coating a resin having a functional group can also be used.

For the base sequence of DNA probes, a base sequence complementary to a part of the base sequence of detection target nucleic acid molecules. For example, for the purpose of identifying causal microorganism of infectious diseases, DNA fragments as amplification products having base sequences corresponding to genome areas coding 16s rRNA of concerned bacteria are used as detection target nucleic acid molecules. That is, DNA probes corresponding to the partial base sequence are designed from base sequences of regions coding rRNA. For the partial base sequence which is used for DNA probes, a base sequence that is characteristic of concerned microorganism and has a very high specificity is preferably selected. It is necessary to ensure that detection target nucleic acid molecules retain all base sequences of regions coding 16s rRNA of concerned microorganism, and a plurality of kinds of DNA probes having corresponding partial base sequences on a plurality of areas included in base sequences of regions coding 16s rRNA of concerned bacteria are designed. At this time, the base lengths and base sequences of individual DNA probes are preferably selected so that a plurality of kinds of DNA probes have no variations “wherever possible” in the probability of forming hybrid bodies with detection target nucleic acid molecules.

For example, when there are a plurality of candidates of causal microorganism of infectious diseases which can be contracted by the patient, for example, a plurality of kinds of DNA probes having corresponding partial base sequences on a plurality of areas included in base sequences of regions coding 16s rRNA of concerned microorganism are designed for each of missed causal microorganism candidates. On the DNA microarray, a plurality of kinds of designed DNA probes capable of being used for identification of candidates of causal microorganism of infectious diseases are fixed in the form of an array for the candidates of causal microorganism of infectious diseases, and as a whole, a two-dimensional matrix in which probe arrays for identification of candidates of causal microorganism of infectious diseases are regularly arranged in a number equivalent to the number of candidates of causal microorganism of infectious diseases.

In the hybridization reaction step 405, the surface of the DNA microarray is washed and a liquid containing detection target nucleic acid molecules is removed after completion of the hybridization reaction. Nucleic acid molecules nonselectively deposited on the surface of the DNA microarray are washed away. If fluorescent labels are used as labels, normally, the washed DNA microarray is dried by draining, and processing proceeds to a next fluorescence measuring step 406. In the fluorescence measuring step 406, the amount of fluorescence from the fluorescence labeling material is measured to quantitatively determine the weight of fluorescence-labeled nucleic acid molecules fixed with hybridization for spots of DNA probes fixed on the DNA microarray. At this time, spots of DNA probes fixed on the DNA microarray are arranged in the form of an array, the entire surface of the DNA microarray is irradiated with excitation light, and the fluorescence intensities at these spots arranged in the form of an array are recorded in the form of a two-dimensional image. That is, in a scan image step 407, a fluorescent light measuring sensor is relatively two-dimensionally scanned while irradiating the entire surface of the DNA microarray with excitation light, and the fluorescence intensities are recorded in the form of two-dimensional image information.

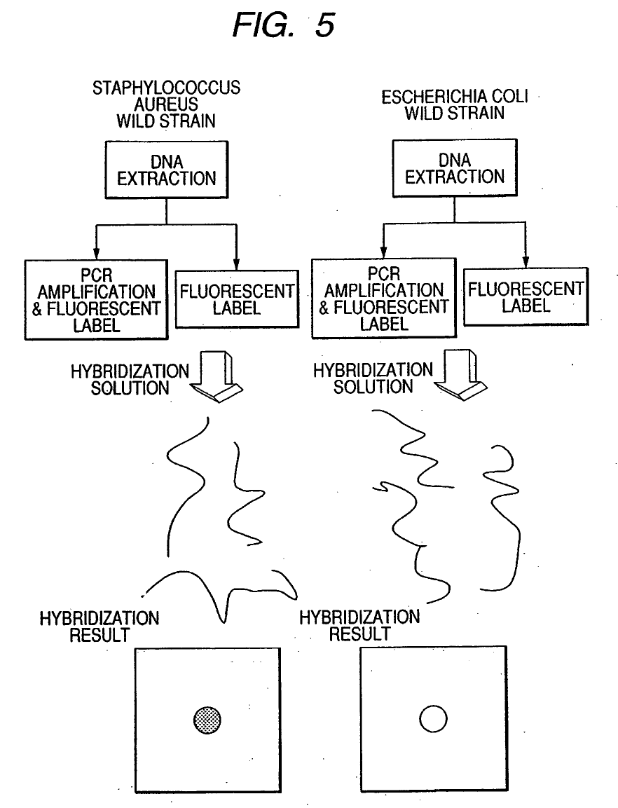

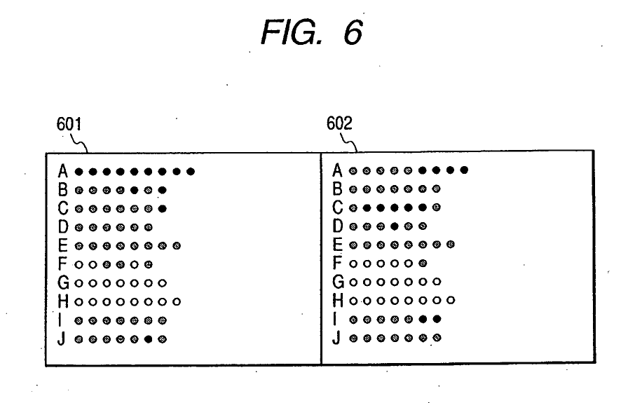

The principle of a method for identifying causal microorganism of an infectious disease using DNA probes fixed on a DNA microarray will now be described. A procedure for distinguishing between Staphylococcus aureus and other bacteria using a DNA microarray fabricated for the purpose of identifying Staphylococcus aureus, and its principle are schematically shown in FIG. 5 as one example.

In FIG. 5, the left line is a detection treatment line for nucleic acid molecules including base sequences of regions coding 16s rRNA originating from a Staphylococcus aureus wild strain, and the right line is a detection treatment line for nucleic acid molecules including base sequences of regions coding 16s rRNA originating from a Escherichia coli wild strain. For example, the left line corresponds to a flow of treating a blood sample taken from a patient infected with Staphylococcus aureus, and the right line corresponds to a flow of treating a blood sample taken from a patient infected with Escherichia coli.

In both the treatment lines, basically same treatments are carried out in the steps of extracting contained DNA from samples taken, amplifying detection target nucleic acid molecules, and labeling the nucleic acid molecules. Namely, first, DNA is extracted from samples taken, for example blood and sputum of a patient infected with bacteria. In this step, every genome DNA contained in samples taken are extracted. In addition to causal microorganism of the infectious disease, cells of the patient, for example various blood cells and body cells can be included in the sample taken, and generally, extracted DNA can include human DNA originating from body cells of the patient.

When the content of detection target nucleic acid molecules originating from causal microorganism of the infectious disease in the total amount of DNA extracted from the sample is low, detection target nucleic acid molecules are amplified using selective amplification means such as the PCR method. In this amplification step, a labeling step of adding as a label a fluorescent material or a material to which a fluorescent material can be coupled in amplification products as a label is generally carried out at the same time.

If amplification is not performed, a fluorescent material or a material to which a fluorescent material can be coupled is directly added to the extracted DNA as a label. Alternatively, the extracted DNA may be used as a mold to fabricate its complementary chain, and in this process, a fluorescent material or a material to which a fluorescent material can be coupled may be added in the elongated DNA chain as a label.

For the purpose of identifying causal microorganism of the disease, a DNA part having a base sequence specific to such microorganism is selected as detection target nucleic acid molecules. For example, the base sequence of ribosome RNA called 16s rRNA which is used for classification of bacteria includes a characteristic base sequence allowing bacteria to be discriminated. Thus, when performing amplification, DNA fragments including base sequences of regions coding 16s rRNA are generally prepared as a PCR amplification product from genome DNA of concerned bacteria.

The species of causal microorganism of the infectious disease contained in a sample taken is usually unknown, and a PCR primer for which DNA fragments including base sequences of regions coding human-originated 16s rRNA are not amplified, but DNA fragments including base sequences of regions coding 16s rRNA originating from many species of microorganism can be amplified at a time for causal microorganism of infectious diseases is used. That is, conditions under which similar partial base sequences in genome DNA among various species of causal microorganism of infectious diseases are used, and corresponding mix primer sets are used to carry out a multiplex PCR reaction to amplify DNA fragments including base sequences of regions coding 16s rRNA originating from a plurality of species of causal microorganism of infectious diseases are selected.

If it is desired to perform more detailed analysis of base sequences for causal microorganism of the identified infectious disease, for example, a PCR primer set specifically for Staphylococcus aureus and a PCR primer set specifically for Escherichia coli are separately selected. If the PCR primer sets specifically for respective bacteria are used, specific areas of the genome DNA of target bacteria can selectively be amplified. In a specimen DNA solution for a hybridization reaction containing a PCR amplification product obtained by the multiplex PCR reaction, the kinds of base sequences of included DNA fragments are quite limited.

In the sample taken, the abundance ratio of causal microorganism of the infectious disease is significantly high, there are not a few cases where microorganism present in the natural world are included although the amount thereof is very small. There are not a few cases where in the specimen DNA solution for a hybridization reaction, DNA fragments originating from included microorganism present in the natural world are included in a slight amount in addition to DNA fragments originating from causal microorganism of the detection target infectious disease as a result of the multiplex PCR reaction. In addition, there are not a few cases where for causal microorganism of the detection target infectious disease, strains present in the natural world does not belong to a single species, but there are several species of strains that have been clinically isolated. A plurality of species of strains may coexist in the sample taken for causal microorganism of the detection target infectious disease. The kinds of base sequences of DNA fragments included in the specimen DNA solution for a hybridization reaction by carrying out PCR amplification are quite limited, but cases where there is only one kind of base sequence of the DNA fragments are rare.

DNA probes designed for the purpose of determination of Staphylococcus aureus form hybrid bodies with single strand DNA including base sequences of regions coding 16s rRNA originating from Staphylococcus aureus, and therefore in the treatment line on the left in FIG. 5, nucleic acid molecules containing labels are observed (positive) at spots of DNA probes for determination of Staphylococcus aureus on the DNA microarray. If DNA probes designed for the purpose of determination of Staphylococcus aureus have very high selectivity, they do not form hybrid bodies with single strand DNA including base sequences of regions coding 16s rRNA originating from a Escherichia coli wild strain, and therefore in the treatment line on the right in FIG. 5, nucleic acid molecules containing labels are not observed (negative) at spots of DNA probes for determination of Staphylococcus aureus on the DNA microarray.

DNA probes designed for the purpose of determination of Escherichia coli form hybrid bodies with single strand DNA including base sequences of regions coding 16s rRNA originating from Escherichia coli, and therefore in the treatment line on the right in FIG. 5, nucleic acid molecules containing labels are observed (positive) at spots of DNA probes for determination of Escherichia coli on the DNA microarray. If DNA probes designed for the purpose of determination of Escherichia coli have very high selectivity, they do not form hybrid bodies with single strand DNA including base sequences of regions coding 16s rRNA originating from Staphylococcus aureus wild strains, and therefore in the treatment line on the right in FIG. 5, nucleic acid molecules containing labels are not observed (negative) at spots of DNA probes for determination of Escherichia coli on the DNA microarray.

If DNA probes designed for the purpose of determination of causal microorganism of various kinds of infectious diseases have very high selectivity, a plurality of kinds of DNA probes for determination of causal microorganism of various kinds of infectious diseases can be spotted in the form of an array on the DNA microarray in accordance with a predetermined arrangement order, and used for making a determination on existence or nonexistence at a time for a plurality of species of causal microorganism of infectious diseases.

Actual operations, their conditions and the like when applying the method and procedure for identifying the species of causal microorganism of infectious diseases contained in samples, described above with FIGS. 4 and 5, will be described in detail below with specific examples.

<Preparation of Probe DNA>

Probes I-1 to I-7 having nucleic acid sequences (I-n) (n is a positive integer) shown in Table 1 were designed as probes for detection of Enterobacter cloacae.

Specifically, the base sequences of probes I-1 to I-7 shown in Table 1 described below are selected from a genome area coding 16s rRNA of concerned bacteria. The base sequences of a group of these probes are designed by selecting their base lengths and the ratio of bases contained so that it can be expected that the base sequences have a very high specificity to concerned bacteria, and at the same time, when they are hybridized with cDNA prepared from regions coding 16s rRNA using genome DNA as a mold, no variations occur in hybridization efficiency among probes “wherever possible”.

TABLE 1

Probe for detection of Enterobacter cloacae:
I-n

| | |
|---|---|
| Probe ID No. | Base sequence |

| | |
|---|---|
| I-1 (SEQ65) | CAgAgAgCTTgCTCTCgggTgA |
| 1-2 (SEQ66) | gggAggAAggTgTTgTggTTAATAAC |
| 1-3 (SEQ67) | ggTgTTgTggTTAATAACcACAgCAA |
| 1-4 (SEQ68) | gCggTCTgTCAAgTCggATgTg |
| 1-5 (SEQ69) | ATTCgAAACTggCAggCTAgAgTCT |
| 1-6 (SEQ70) | TAACCACAgCAATTgACgTTACCCg |
| 1-7 (SEQ71) | gCAATTgACgTTACCCgCAgAAgA |

After the DNA probes are chemically synthesized, a thiol group is introduced at the 5′ terminal of the nucleic acid chain as a functional group for use in fixation to the surface of a solid phase on the DNA microarray in accordance with an established method. After the functional group is introduced at the 5′ terminal, the DNA probes are purified and freeze-dried. The freeze-dried DNA probes for detection of Enterobacter cloacae are stored in a freezer at −30° C.

For detection of various kinds of disease-causing microorganism, probes having a very high specificity to concerned microorganism are designed from genome areas coding 16s rRNA originating from respective microorganism using a similar method. Base sequences selected as DNA probes for detection of Staphylococcus aureus: A-n, DNA probes for detection of Staphylococcus epidermidis: B-n, DNA probes for Escherichia coli: C-n, DNA probes for detection of Klebsiella pneumoniae: D-n, DNA probes for detection of Pseudomonas aeruginosa: E-n, DNA probes for detection of Serratia: F-n, DNA probes for detection of Streptococcus pneumoniae: G-n, DNA probes for detection of Haemophilus influenzae: H-n, and DNA probes for detection of Enterococcus faecalis: J-n (n is a positive integer) are shown in Tables 2 to 10, respectively.

TABLE 2

DNA probes for detection of Staphylococcus
aureus: A-n

| | |
|---|---|
| Probe ID No. | Base sequence |

| | |
|---|---|
| A-1 (SEQ7) | gAAccgCATggTTCAAAAgTgAAAgA |
| A-2 (SEQ8) | CACTTATAgATggATCCgCgCTgC |
| A-3 (SEQ9) | TgCACATCTTgACggTACCTAATCAg |
| A-4 (sEQ10) | ccccTTAgTgcTgcAgcTAAcg |
| A-5 (SEQ11) | AATACAAAgggcAgCgAAACCgC |
| A-6 (SEQ12) | CCggTggAgTAACCTTTTAggAgCT |
| A-7 (SEQ13) | TAACCTTTTAggAgCTAgCCgTCgA |
| A-8 (SEQ14) | TTTAggAgCTAgCCgTCgAAggT |
| A-9 (SEQ15) | TAgCCgTCgAAggTgggACAAAT |

TABLE 3

DNA probes for detection of Staphylococcus
epidermidis: B-n

| | |
|---|---|
| Probe ID No. | Base sequence |

| | |
|---|---|
| B-1 (SEQ16) | gAACAgACgAggAgCTTgCTCC |
| B-2 (SEQ17) | TAgTgAAAgACggTTTTgCTgTCACT |
| B-3 (SEQ18) | TAAgTAACTATgCACgTCTTgACggT |
| B-4 (SEQ19) | gACCCCTCTAgAgATAgAgTTTTCCC |
| B-5 (SEQ20) | AgTAACCATTTggAgCTAgCCgTC |
| B-6 (SEQ21) | gAgCTTgCTCCTCTgACgTTAgC |
| B-7 (SEQ22) | AgCCggTggAgTAACCATTTgg |

TABLE 4

DNA probes for Escherichia coli: C-n

| | |
|---|---|
| Probe ID No. | Base sequence |

| | |
|---|---|
| C-1 (SEQ23) | CTCTTgCCATCggATgTgCCCA |
| C-2 (SEQ24) | ATACCTTTgCTCATTgACgTTACCCg |
| C-3 (SEQ25) | TTTgCTCATTgACgTTACCCgCAg |
| C-4 (SEQ26) | ACTggCAAgCTTgAgTCTCgTAgA |
| C-5 (SEQ27) | ATACAAAgAgAAgCgACCTCgCg |
| C-6 (SEQ28) | CggACCTCATAAAgTgCgTCgTAgT |
| C-7 (SEQ29) | gCggggAggAAgggAgTAAAgTTAAT |

TABLE 5

DNA probes for detection of Klebsiella
pneumoniae: D-n

| | |
|---|---|
| Probe ID No. | Base sequence |

| | |
|---|---|
| D-1 (SEQ30) | TAgCACAgAgAgCTTgCTCTCgg |
| D-2 (SEQ31) | TCATgCCATCAgATgTgCCCAgA |
| D-3 (SEQ32) | CggggAggAAggCgATAAggTTAAT |
| D-4 (SEQ33) | TTCgATTgACgTTACCCgcAgAAgA |
| D-5 (SEQ34) | ggTCTgTCAAgTCggATgTgAAATCC |
| D-6 (SEQ35) | gCAggCTAgAgTCTTgTAgAgggg |

TABLE 6

DNA probes for detection of Pseudomonas
aeruginosa: E-n

| | |
|---|---|
| Probe ID No. | Base sequence |

| | |
|---|---|
| E-1 (SEQ36) | TgAgggAgAAAgTgggggATCTTC |
| E-2 (SEQ37) | TCAgATgAgCCTAggTCggATTAgC |
| E-3 (SEQ38) | gAgCTAgAgTACggTAgAgggTgg |
| E-4 (SEQ39) | gTACggTAgAgggTggTggAATTTC |
| E-5 (SEQ40) | gACCACCTggACTgATACTgACAC |
| E-6 (SEQ41) | TggCCTTgACATgCTgAgAACTTTC |
| E-7 (SEQ42) | TTAgTTACCAgCACCTCgggTgg |
| E-8 (SEQ43) | TAgTCTAACCgCAAgggggACg |

TABLE 7

DNA probes for detection of Serratia: F-n

| | |
|---|---|
| Probe ID No. | Base sequence |

| | |
|---|---|
| F-1 (SEQ44) | TAgCACAgggAgCTTgCTCCCT |
| F-2 (SEQ45) | AggTggTgAgCTTAATACgCTCATC |
| F-3 (SEQ46) | TCATCAATTgACgTTACTCgCAgAAg |
| F-4 (SEQ47) | ACTgCATTTgAAACTggCAAgCTAgA |
| F-5 (SEQ48) | TTATCCTTTgTTgCAgCTTCggCC |
| F-6 (SEQ49) | ACTTTCAgCgAggAggAAggTgg |

TABLE 8

DNA probes for detection of Streptococcus
pneumoniae: G-n

| | |
|---|---|
| Probe ID No. | Base sequence |

| | |
|---|---|
| G-1 (SEQ50) | AgTAgAAcgCTgAAggAggAgcTTg |
| G-2 (SEQ51) | CTTgCATCACTACCAgATggACCTg |
| G-3 (SEQ52) | TgAgAgTggAAAgTTCAcAcTgTgAC |
| G-4 (SEQ53) | gCTgTggCTTAACCATAgTAggCTTT |
| G-5 (SEQ54) | AAgcggcTcTcTggcTTgTAAcT |
| G-6 (SEQ55) | TAgACCCTTTCCggggTTTAgTgC |
| G-7 (SEQ56) | gACggCAAgCTAATCTCTTAAAgCCA |

TABLE 9

DNA probes for detection of Haemophilus
influenzae: H-n

| | |
|---|---|
| Probe ID No. | Base sequence |

| | |
|---|---|
| H-1 (SEQ57) | gCTTgggAATCTggCTTATggAgg |
| H-2 (SEQ58) | TgCCATAggATgAgCCCAAgTgg |
| H-3 (SEQ59) | CTTgggAATgTACTgACgCTCATgTg |
| H-4 (SEQ60) | ggATTgggCTTAgAgCTTggTgC |
| H-5 (SEQ61) | TACAgAgggAAgCgAAgCTgCg |
| H-6 (SEQ62) | ggCgTTTACCACggTATgATTCATgA |
| H-7 (SEQ63) | AATgCCTACCAAgCCTgCgATCT |
| H-8 (SEQ64) | TATcggAAgATgAAAgTgcgggAcT |

TABLE 10

DNA probes for Enterococcus faecalis: J-n

| | |
|---|---|
| Probe ID No. | Base sequence |

| | |
|---|---|
| J-1 (SEQ72) | TTCTTTCCTCCCgAgTgCTTgCA |
| J-2 (SEQ73) | AACACgTgggTAACCTACCCATCAg |
| J-3 (SEQ74) | ATggCATAAgAgTgAAAggCgCTT |
| J-4 (SEQ75) | gACCCgCggTgCATTAgCTAgT |
| J-5 (SEQ76) | ggACgTTAgTAACTgAACgTCCCCT |
| J-6 (SEQ77) | CTCAACCggggAgggTCATTgg |
| J-7 (SEQ78) | TTggAgggTTTCCgCCCTTCAg |

<Preparation of PCR Primer for Amplification of Specimen DNA>

For detection of ten species of disease-causing microorganism described above, regions coding 16s rRNA (target nucleic acid) originating from concerned bacteria are amplified using a genome DNA as a mold. At this time, primers having nucleic acid sequences shown in Table 11 were designed as PCR primers for amplification which can be used in common for various kinds of disease-causing microorganism.

Specifically, to selectively amplify only regions coding 16s rRNA having a base length of about 1500 on the genome DNA, base sequences of which the melting temperatures are uniform wherever possible are selected when forming hybrid bodies with the mold DNA based on partial base sequences showing high commonality at both end parts of the coding regions of ten species of disease-causing microorganism described above. Three kinds of primers including variants were designed for each primer so that variants, and a plurality of 16s rRNA coding regions present on the genome can be amplified at the same time.

Table 11

PCR primer for amplification of regions
coding 16s rRNA

| | |
|---|---|
| Primer No. | Base sequence |

| | | |
|---|---|---|
| Forward | F-1 (SEQ1) | 5′ GCGGCGTGCCTAATACATGCAAG 3′ |
| Primer | F-2 (SEQ2) | 5′ GCGGCAGGCCTAACACATGCAAG 3′ |
| | F-3 (SEQ3) | 5′ GCGGCAGGCTTAACACATGCAAG 3′ |
| Reverse | R-1 (SEQ4) | 5′ ATCCAGCCGCACCTTCCGATAC 3′ |
| Primer | R-2 (SEQ5) | 5′ ATCCAACCGCAGGTTCCCCTAC 3′ |
| | R-3 (SEQ6) | 5′ ATCCAGCCGCAGGTTCCCCTAC 3′ |

The primers shown in Table 11 are purified by high speed liquid chromatography (HPLC) after they are synthesized. A primer solution was prepared by mixing three kinds of forward primers and three kinds of reverse primers and dissolving the resultant mixture in a TE buffer solution so that each primer had a final concentration of 10 pmol/μl.

<Extraction of Bacterial Genome>

(Pretreatment for Extraction of Genome)

1.0 ml (OD₆₀₀=0.7) of solution containing microorganisms is put in a microtube having a volume of 1.5 ml, and centrifuged (8500 rpm, 5 min, 4° C.) to collect bacterial bodies. The supernatant is discarded, 300 μl of enzyme buffer (50 mM Tris-HCl:pH 8.0, 25 mM EDTA) is then added, and the microbial bodies are resuspended using a mixer. The resuspended bacteria solution is centrifuged again (8500 rpm, 5 min, 4° C.) to collect bacterial bodies. The supernatant is discarded, the following dissolved enzyme solution is then added to the collected bacterial bodies, and the resultant mixture is resuspended using a mixer.

Lysozyme 50 μl (20 mg/ml in enzyme buffer)

N-Acetylmuramidase SG 50 μl (0.2 mg/ml in enzyme buffer)

The bacteria solution obtained by adding the dissolved enzyme solution and resuspending microbial bodies is left standing in an incubator at 37° C. for 30 minutes to carry out processing of dissolution of cell walls.

(Extraction of Genome)

After processing of dissolution is carried out, genome DNA extraction of the microorganisms is carried out using a commercial available nucleic acid purifying kit (MagExtractor−Genome—: manufactured by TOYOBO CO., LTD.) in accordance with the procedures described below.

Step 1

First, after the processing of dissolution is carried out, 750 μl of dissolving and adsorbing solution and 40 μl of magnetic bead solution are added to microorganism suspensions, and the resultant mixture is vigorously stirred for 10 minutes using a tube mixer. By this operation, genome DNA is adsorbed onto the surfaces of magnetic bead particles.

Step 2

Next, a microtube is set in a separating stand (magical trapper), and left standing for 30 seconds to collect magnetic bead particles on the wall surface of the tube. The supernatant is then discarded in a state of being set in the stand.

Step 3

Next, 900 μl of washing solution is added, and the resultant mixture is stirred for about 5 seconds to resuspend magnetic bead particles using a mixer.

Step 4

The microtube is set in the separating stand (magical trapper) again, and left standing for 30 seconds to collect magnetic bead particles on the wall surface of the tube. The supernatant is then discarded in a state of being set in the stand.

Step 5

The steps 3 and 4 are repeated, and second washing is carried out.

Step 6

900 μl of 70% ethanol is added, and the resultant mixture is stirred for 5 seconds by a mixer to resuspend magnetic bead particles.

Step 7

Next, the microtube is set in the separating stand (magical trapper), and left standing for 30 seconds to collect magnetic bead particles on the wall surface of the tube. The supernatant is then discarded in a state of being set in the stand.

Step 8

The steps 6 and 7 are repeated, and second washing with 70% ethanol is carried out.

Step 9

100 μl of pure water is added to the collected magnetic bead particles, and the resultant mixture is stirred for 10 minutes by the tube mixer. By this operation, genome DNA adsorbed on the surfaces of magnetic bead particles is eluted.

Step 10

Next, the microtube is set in the separating stand (magical trapper), and left standing for 30 seconds, and magnetic bead particles are collected on the wall surface of the tube. The supernatant containing dissolved genome DNA is then collected in a new tube in a state of being set in the stand.

(Inspection of Quality of Collected Genome DNA)

Genome DNA of collected microorganisms is subjected to agarose electrophoresis and spectrophotometry at 260 nm/280 nm in accordance with an established method to perform inspection of the quality (the amount of included low-molecular nucleic acid and degree of decomposition) and calculation of the amount (content) of collected DNA.

The collected genome DNA is dissolved in a TE buffer solution so that it has a final concentration of 50 ng/μl, and the resultant solution is used for a mold DNA solution in a PCR amplification reaction described below.

In this example, neither degradation of genome DNA nor inclusion of rRNA was found.

<Fabrication of DNA Microarray>

(1) Washing of Glass Substrate

A glass substrate (size: 25 mm (W)×75 mm (L)×1 mm (T), manufactured by IIYAMA PRECISION GLASS CO., LTD.) made of synthetic silica is put in a heat-resistant and alkali-resistant rack, and immersed in an ultrasonic washing solution adjusted to have a predetermined concentration. The glass substrate is immersed in the washing solution overnight, and then ultrasonically washed for 20 minutes. Subsequently, the substrate is taken out, briefly rinsed with pure water, and then ultrasonically washed in ultra pure water for 20 minutes. Next, the substrate is immersed in for 10 minutes in 1 N aqueous sodium hydroxide heated to 80° C. The substrate is washed with pure water and ultrasonically washed in ultra pure water again to obtain a washed silica glass substrate for fabrication of a DNA microarray chip.

(2) Surface Treatment

A silane coupling agent KBM-603 (Shin-Etsu Silicone Co., Ltd.) is dissolved in pure water so as to have a concentration of 1%, and the resultant solution is stirred at room temperature for 2 hours. Subsequently, the washed silica glass substrate is immersed in the aqueous silica coupling agent, and left standing at room temperature for 20 minutes. The silica glass substrate is taken out, the surface of the substrate is briefly rinsed with pure water, and a nitrogen gas is then blown to both surfaces of the substrate to perform blow drying. Next, the substrate dried by blowing nitrogen is baked for 1 hour in an oven heated to 120° C. to complete a coupling agent treatment. By the coupling agent treatment, an amino group originating from an amino silane coupling agent is introduced onto the surface of the silica glass substrate.

An EMCS solution obtained by dissolving N-maleimidocaproyloxysuccinimide (N-(6-maleimidocaproyloxy) succinimide; hereinafter abbreviated as EMCS) manufactured by Dojindo Laboratories in a mixed solvent of dimethyl sulfoxide and ethanol (1:1) so as to have a final concentration of 0.3 mg/ml is prepared. The baked silica glass substrate is left standing to cool, and immersed in the prepared EMCS solution at room temperature for 2 hours. By this treatment, an amino group introduced onto the surface by the silane coupling agent treatment reacts with a succinimide group of the EMCS to introduce a maleimide group onto the surface of the silica glass substrate. The silica glass substrate taken out from the EMCS solution is washed using the mixed solvent of dimethyl sulfoxide and ethanol (1:1), further washed with ethanol, and then dried under a nitrogen gas atmosphere.

(3) Probe DNA

The probes for detection of disease-causing microorganism fabricated as described above are dissolved in pure water, and the resultant solution is dispensed so as to have a final concentration of 10 μM (when ink is dissolved), and then freeze-dried to remove water.

(4) Ejection of Probe DNA by BJ Printer and Coupling to Substrate

An aqueous solution containing 7.5 wt % of glycerin, 7.5 wt % of thiodiglycol, 7.5 wt % of urea and 1.0 wt % of Acetylenol EH (manufactured by Kawaken Fine Chemicals Co., Ltd.) is prepared. Subsequently, seven kinds of probes I-1 to I-7 (Table 1) prepared previously are dissolved in the aqueous solution so as to have a normal concentration. The obtained DNA solution is filled in an ink tank for a bubble jet printer (trade name: BJF-850, manufactured by Canon Inc.), and the ink tank is attached to a print head.

In this connection, the bubble jet printer which is used here has been modified so as to allow printing onto a flat plate. The bubble jet printer has an apparatus configuration such that droplets of about 5 picoliters of DNA solution can be spotted at pitches of about 120 micrometers by inputting a print pattern in accordance with a predetermined file creation method.

Subsequently, using the modified bubble jet printer, a print operation is performed on the surface of one silica glass substrate to fabricate a DNA microarray. After confirming that each DNA solution has reliably been spotted, the DNA microarray is left standing in a moistening chamber for 30 minutes to allow the maleimide group on the surface of the silica glass substrate to react with the thiol group at the 5′ terminal of the DNA probe.

(5) Washing

After the reaction for 30 minutes, a solution containing unreacted DNA remaining on the surface is washed away by a 10 mM phosphate buffer solution (pH 7.0) containing 100 mM of NaCl. As a result, a DNA microarray chip having single strand DNA for probes fixed in the form of an array on the surface of the silica glass substrate is obtained.

<Amplification and Labeling of Specimen DNA (PCR Amplification & Capture of Fluorescent Labels)>

Using as a mold the genome DNA extracted from microorganisms taken from the specimen, amplification of regions coding 16s rRNA (target nucleic acid) originating from concerned bacteria and labeling of amplification products using fluorescent labels at this time are carried out under the PCR reaction conditions described below.

TABLE 12
Table 12 Composition of PCR reaction solution

| | Compounding | Final |
|---|---|---|
| Composition | amount μL | concentration |
| Premix PCR reagent (TAKARA | 25.0 | |
| ExTaq) | | |
| Template Genome DNA | 2.0 | 100 ng/50 μL |
| Forward Primer mix | 2.0 | 20 pmol/tube each |
| Reverse Primer mix | 2.0 | 20 pmol/tube each |
| Cy-3 dUTP (1 mM) | 2.0 | 2 nmol/tube |
| H₂O | 17.0 | |
| Total | 50.0 | |

TABLE 13
Table 13 Temperature condition for PCR reaction

| Cycle | Temperature | | |
|---|---|---|---|
| operation | ° C. | Time period | |
| Denature | 95 | 10 minutes | |
| Denature | 92 | 45 seconds | |
| Anneal | 55 | 45 seconds | 35 cycles |
| Extention | 72 | 45 seconds | |
| Extention | 72 | 10 minutes | |
| Store | 10 | Overnight (14 hours) | |

The aforesaid temperature cycle is accomplished using a commercially available thermal cycler.

After the PCR amplification reaction, a primer is removed using a commercially available purifying column (QIAGEN QIAquick PCR Purification Kit), and amplification products are then quantitatively determined. The obtained amplification product is labeled with fluorescence originating from Cy-3 dUTP, and used as labeled specimen DNA in the hybridization reaction described below.

<Hybridization>

The DNA microarray chip fabricated in accordance with the procedure of “Fabrication of DNA Microarray” and labeled specimen DNA prepared in accordance with the procedure of “Amplification and Labeling of Specimen DNA (PCR Amplification & Capture of Fluorescent Label) are used to detect specimen DNA by a probe hybridization reaction.

(Blocking of DNA Microarray)

BSA (bovine serum albumin fraction V: manufactured by Sigma) is dissolved in a 100 mM NaCl/10 mM phosphate buffer so as to have a concentration of 1 wt %. The DNA microarray chip fabricated in accordance with the procedure of “Fabrication of DNA Microarray” is immersed in the BSA solution at room temperature for 2 hours, and subjected to a blocking treatment. After completion of the blocking treatment, the DNA microarray chip is washed with a 2×SSC solution (300 mM of NaCl and 30 mM of sodium citrate (trisodium citrate dihydrate, C₆H₅Na₃.2H₂O), pH 7.0) containing 0.1 wt % SDS (dodecyl sodium sulfate). Further, the DNA microarray chip is rinsed with pure water, and drained by a spin drying apparatus.

(Hybridization)

The drained DNA microarray chip is set in a hybridization apparatus (Genomic Solutions Inc., Hybridization Station), and a hybridization reaction is made to proceed under the conditions shown below.

(Hybridization Solution)

6×SSPE/10% formamide/Target (2nd PCR products, total amount)

(6×SSPE: 900 mM of NaCl, 60 mM of NaH₂PO₄.H₂O and 6 mM of EDTA, pH 7.4)

(Hybridization Conditions)

65° C., 3 min->92° C., 2 min->45° C., 3 hr->Wash 2×SSC/0.1% SDS at 25° C.->Wash 2×SSC at 20° C.->(Rinse with H₂O: Manual)->Spin dry

<Detection of Target DNA Originating from Microorganism (Fluorometry)>

After completion of the hybridization reaction, fluorescent intensities at spots on the drained DNA microarray chip are measured using a fluorescence detecting apparatus for a DNA microarray (GenePix 4000B manufactured by Axon Co., Ltd.). At this time, the fluorescent intensities at spots on the DNA microarray are recorded in the form of two-dimensional image information.

One example of a two-dimensional image representing fluorescent intensities at spots on the DNA microarray is shown in FIG. 6. In FIG. 6, colors representing the fluorescent intensities associated with fluorescence-labeled DNA forming hybrid bodies with DNA probes fixed at the spots become darker as their intensities increase. That is, colors representing the fluorescent intensities become darker as the amount of fluorescence-labeled DNA forming hybrid bodies with DNA probes fixed at the spots increases.

On the DNA microarray illustrated in FIG. 6, DNA probes for detection of ten species of microorganisms described in “Preparation of Probe DNA”, i.e. Staphylococcus aureus: A-n, Staphylococcus epidermidis: B-n, Escherichia coli: C-n, Klebsiella pneumoniae: D-n, Pseudomonas aeruginosa: E-n, Serratia: F-n, Streptococcus pneumoniae: G-n, Haemophilus influenzae: H-n, Enterobacter cloacae: I-n and Enterococcus faecalis: J-n are spotted in the form of an array respectively.

A specimen sample containing amplification products of regions coding 16s rRNA (target nucleic acid) originating from concerned bacteria is made to react with probe DNA fixed on the DNA microarray using as a mold the genome DNA extracted from various kinds of disease-causing microorganism. In FIG. 6, a scan image 601 of the DNA microarray on the left is one example of a two-dimensional image observed when a specimen sample containing amplification products of regions coding 16s rRNA (target nucleic acid) originating from Staphylococcus aureus is made to undergo a reaction, and a scan image 602 of the DNA microarray on the right is one example of a two-dimensional image observed when a specimen sample containing amplification products of regions coding 16s rRNA (target nucleic acid) originating from Escherichia coli is made to undergo a reaction.

In the scan image 601 of the DNA microarray, fluorescent intensities associated with fluorescent labels at spots of DNA probes for detection of Staphylococcus aureus: A-n are high, while in the scan image 602 of the DNA microarray, fluorescent intensities associated with fluorescent labels at spots of DNA probes for detection of Escherichia coli: C-n are high. Ideally, base sequences of DNA probes are selected so that a plurality of kinds of DNA probes for detection of disease-causing microorganism form hybrid bodies with only amplification products of regions coding 16s rRNA (target nucleic acid) originating from concerned bacteria, and does not form hybrid bodies with amplification products of regions coding 16s rRNA (target nucleic acid) originating from other bacteria.

Actually, however, a plurality of kinds of DNA probes for detection of disease-causing microorganism should be selected so as to cover all of amplification products of regions coding 16s rRNA (target nucleic acid) originating from concerned bacteria, and some DNA probes have base sequences capable of misfit hybridization with amplification products of regions coding 16s rRNA (target nucleic acid) originating other bacteria. That is, as a result of occurrence of so called a “cross hybridization reaction”, amplification products of regions coding 16s rRNA (target nucleic acid) originating from detection target disease-causing microorganism form hybrid bodies with some of DNA probes for detection of other bacteria. In the scan image 601, there are some spots at which fluorescent intensities associated with fluorescent labels are observed to be high, in addition to the spots of DNA probes for detection of Staphylococcus aureus: A-n. Similarly, in the scan image 602, there are some spots at which fluorescent intensities associated with fluorescent labels are observed to be high, in addition to the spots of DNA probes for detection of Escherichia coli: C-n.

Close comparison of fluorescent intensities at the spots of DNA probes for detection of Escherichia coli: C-n in the scan image 602 shows that some of the spots are inferior in fluorescent intensity to other spots. When elongating the DNA chain as means for adding fluorescent labels to amplification products of regions coding 16s rRNA (target nucleic acid) originating from detection target disease-causing microorganism, a method of coupling Cy-3 dU in place of T is used, and therefore the efficiency of hybridization of an area including Cy-3 dU and DNA probes significantly decreases as compared to a case where original T is included. Some of amplification products of regions coding 16s rRNA (target nucleic acid) originating from detection target disease-causing microorganism do not include fluorescent labels to which Cy-3 dU is not coupled in place of T. Therefore, for some base sequences of DNA probes, formation of hybrid bodies with DNA fragments to which fluorescent labels are actually added is less dominant, while formation of hybrid bodies with DNA fragments to which fluorescent labels are not added is relatively dominant. In this case, there is no significant difference in the total amount of hybrid bodies formed at spots of DNA probes, but fluorescent intensities associated with fluorescent labels observed at the spots are low.

In addition, for individual disease-causing microorganism, clinically isolated strains belong to the same species, but there are not a few cases where the strains correspond to variant strains having some variants for typical wild strains based on base sequences of DNA probes for detection of concerned disease-causing microorganism in designing the DNA probes. Base sequences of regions coding 16s rRNA (target nucleic acid) originating from such variant strains are almost same as base sequences reported with typical wild strains, but there are not a few cases where a little variants exist. Therefore, there are not a few cases where there is a difference in the pattern of fluorescent intensities observed at spots of DNA probes on the DNA microarray between amplification products prepared from clinically isolated strains and amplification products prepared from typical wild strains.

Particularly, regarding disease-causing microorganism, various kinds of resistant strains have emerged as a result of application of therapeutic methods for inhibiting the growth of the bacteria by administration of antibiotics in clinical fields. Under such a circumstance, in addition to identification of the species of disease-causing microorganism existing in samples taken from a new patient, identification of their strains from various kinds of resistant strains is required. When identification of the strain is required, not only a pattern of fluorescent intensities observed at spots of DNA probes on the DNA microarray in amplification products prepared from typical wild strains, but also close contrast with a pattern of fluorescent intensities for amplification products prepared from various kinds of resistant strains observed in past cases is effective.

In the electronic clinical chart system according to the present invention, a pattern of fluorescent intensities observed at spots of DNA probes on the DNA microarray in amplification products prepared from disease-causing microorganism existing in samples taken from patients in the database of examination data included in electronic clinical charts of a large number of patients as past cases can be contrasted with a pattern of fluorescent intensities observed in samples taken from a new patient to search for past cases having a similar pattern of fluorescent intensities. A doctor in charge of the diagnosis and treatment of a new patient refers to the diagnosis result, the reason for the diagnosis, the symptom of the patient and the records of treatments conducted on the patient, described in the electronic clinical chart of the patient, for a past case of which the pattern of fluorescent intensities is most similar to that for the new patient to considerably facilitate the diagnosis and determination of a treatment policy for the new patient whom the doctor is responsible for.

For example, if the strain of disease-causing microorganism with which the new patient is infected is similar to the strain of disease-causing microorganism with which similar case patients selected by the search were infected in the pattern of fluorescent intensities observed at spots of DNA probes on the DNA microarray, but it turns out that the strain shows decisive differences, it can be presumed that the strain is likely to be a new resistant strain that is not included in conventional cases. In this case, it can quickly be determined that further detailed examinations should be conducted.

If there are a plurality of species of causal microorganism that are involved in causing the symptom of the new patient, it is difficult to make a proper diagnosis only by referring to a typical pattern of fluorescent intensities observed in infection with one species of causal microorganism. In this case, finding past cases where a plurality of species of causal microorganism were involved by searching for past cases having a similar pattern of fluorescent intensities considerably facilitates a proper diagnosis.

A pattern of fluorescent intensities measured in the patient and observed at spots of DNA probes on the DNA microarray is expressed as, for example, a multidimensional vector having fluorescent intensities observed at the spots as elements. For example, as for the DNA microarray shown in FIG. 6, the pattern is expressed as a 72-dimensional vector {x₁, . . . , X₇₂} having fluorescent intensities (Xᵢ) observed at the spots as elements in relation to 72 kinds of DNA probes in total, i.e. A-1 to A-9, B-1 to B-7, C-1 to C-7, D-1 to D-6, E-1 to E-8, F-1 to F-6, G-1 to G-7, H-1 to H-8, I-1 to I-7 and J-1 to J-7.

A vector Xₙ{X₁ₙ, . . . , X₇₂ₙ} corresponding to the results of examinations of the new patient is contrasted with a vector Xⱼ{X₁ⱼ, . . . , X₇₂ⱼ) corresponding to the results of examinations of patients as past cases stored in the electronic clinical chart system, and past cases (patients) showing a similar vector Xⱼ{X₁ⱼ, . . . , X₇₂ⱼ} are selected. For example, in consideration of a difference between two vectors; ΔXₙⱼ=Xₙ−Xⱼ={(X₁ₙ−Xᵢⱼ), . . . , (X₇₂ₙ−X₇₂ⱼ)}, it is determined that the similar vector Xⱼ(X₁ⱼ, . . . , X₇₂ⱼ} has a small magnitude of ΔXₙⱼ. That is, it can be determined that the similarity becomes higher as the magnitude of ΔXₙⱼ: {(X₁ₙ−X₁ⱼ)²+ . . . +(X₇₂ₙ−X₇₂ⱼ)²}^{1/2} decreases. The magnitude of ΔXₙⱼ: {(X₁ₙ−X₁ⱼ)²+ . . . +(X₇₂ₙ−X₇₂ⱼ)²}^{1/2} is calculated for all vectors Xⱼ{X₁ⱼ, . . . , X₇₂ⱼ} corresponding to the results of examinations of certain patients as past cases stored in the electronic clinical chart system, and past cases (patients) are then sorted in ascending order of the magnitude of ΔXₙⱼ. That is, this is a method in which the level of similarity is determined using as an indicator the magnitude of ΔXₙⱼ: {(X₁n−X₁ⱼ)²+ . . . +(X₇₂ₙ−X₇₂ⱼ)²}^{1/2} corresponding to the Euclidean distance between two vectors.

When the magnitude of ΔXₙⱼ: {(X₁n−X₁ⱼ)²+ . . . +(X₇₂ₙ−X₇₂ⱼ)²}^{1/2} is used as an indicator, the magnitude of ΔXₙⱼ is not determined to be 0 even if, for example, the vector Xₙ{X₁ₙ, . . . , X₇₂ₙ} corresponding to the results of examinations of the new patient and the vector Xⱼ{X₁ⱼ, . . . , X₇₂ⱼ} corresponding to the results of examinations of certain past cases (patients) meet the requirement of Xₙ=kXⱼ. That is, for the results of actual measurement, the fluorescent intensities may relatively vary due to systematic errors. For measured data, there is no means for making a correction for such relative variations, and therefore uncorrected measurement results are stored. In some cases, a past case which would fully coincide if a correction had been made for relative variations in intensity could not be searched as a most similar past case (patient).

In consideration of this respect, it is more preferable in many cases that standardization is performed for the vector Xₙ{X₁ₙ, . . . , X₇₂ₙ} and the vector Xⱼ{X₁ⱼ, . . . , X₇₂ⱼ} which are contrasted with each other, and a difference between two vectors: ΔXₙⱼ≡Xₙ−Xⱼ={(X₁ₙ−Xᵢⱼ), . . . , (X₇₂ₙ−X₇₂ⱼ)} is then considered.

Alternatively, the level of similarity can be determined using as an indicator an angle formed by two vectors. Specifically, the angle θ formed by two vectors is defined using the relational expression of Xₙ·Xⱼ≡Xₙ·Xⱼ cos θ, from the inner product Xₙ·Xⱼ≡{(X₁ₙ·X₁ⱼ)+ . . . +(X₇₂ₙ·X₇₂ⱼ)} and the magnitudes of two vectors: Xₙ≡{(X₁ₙ)²+ . . . +(X₇₂ₙ)²}^{1/2} and Xⱼ≡{(X₁ⱼ)²+ . . . +(X₇₂ⱼ)²}^{1/2}. In this method, it is determined that the level of similarity between two vectors becomes high as the angle θ formed by directions along which two vectors are oriented becomes small. The method in which the angle θ formed by directions along which two vectors are oriented is considered is superior in terms of principle although there is no substantial difference as compared to the aforementioned method in which standardization is performed in advance and a difference between two vectors: ΔXₙⱼ is considered.

As a measure for the “difference” between two vectors, a measure other than the aforementioned indicator may be used.

As described above, examination data (primary data) can be formed into numerical data in principle according to examination items. Thus, for various examination items, the degree of similarity between data can be determined by integrating examination results formed into numerical data to handle them as one vector, and determining the degree of similarity between vectors.

Symptoms (clinical finding data) are not normally formed into numerical data, but can be formed into numerical data by giving scores on presence/absence of symptoms and the degree of seriousness. Alternatively, the form of description of symptoms (clinical finding data) on the electronic clinical chart can be a form of description based on preset criteria for giving scores on presence/absence of symptoms and the degree of seriousness. That is, by giving scores on presence/absence of symptoms and the degree of seriousness, the degree of similarity can be determined by a similar method for symptoms (clinical finding data).

For example, after extracting preset areas corresponding to “keywords” showing symptoms for descriptions of symptoms (clinical finding data) written in a normal writing style by applying a character search technique, scores can be given on presence/absence of symptoms and the degree of seriousness. Specifically, a function of comparing symptoms can be implemented by incorporating a published technique such as a full text search system Namazu (http://www.namazu.org/) into a system.

Examination data and symptoms (clinical finding data) both reflect conditions of diseases of patients resulting from a certain disease cause, but correspond to conditions of diseases of patients projected onto different coordinate planes. For example, symptoms (clinical finding data) change with time as conditions of diseases of patients progress. Therefore, the degree of progress of disease conditions in a new patient is considered, and then an inclusive similarity degree inclusively considering two aspects of examination data and symptoms (clinical finding data) is calculated. For example, after calculating the similarity degree of examination data: ΔX and the similarity degree of symptoms: ΔY, weighed coefficients a and b can be added to the similarity degrees to calculate the inclusive similarity degree as {aΔX+bΔY}. For example, if a=0 is selected for the weighed coefficient a for the similarity degree of examination data, the search function corresponds to a search function used in the conventional electronic clinical chart system in which similar past cases are searched based solely on symptoms (clinical finding data). Generally, the function of searching past similar case patients which is used in the electronic clinical chart system of the present invention is configured to consider both of the similarity degree of examination data: ΔX and the similarity degree of symptoms: ΔY, and therefore the weighed coefficient a for the similarity degree of examination data is preferably selected at least equivalently to the weighed coefficient b for the similarity degree of symptoms: ΔY. For example, by selecting the ratio of weighed coefficient a:weighed coefficient b to be about a:b=5:1 to 1:2, search of past similar case patients pacing more importance on the similarity degree of examination data can be conducted.

The electronic clinical chart system according to the present invention can suitably be used, for example, when a doctor in charge searches similar case patients showing a symptom and the results of examinations similar to those of a new patient from past cases stored in the electronic clinical chart system, and refers to the symptom and the results of examinations of the similar case patients to conduct a diagnosis when making a diagnosis for the disease cause of the new patient, whereby a more accurate diagnosis is made.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2005-125252 filed Apr. 22, 2005, which is hereby incorporated by reference herein.

What is claimed is:

1. An electronic clinical chart system comprising:
a patient electronic clinical chart database storing patients' examination data, clinical finding data and treatment data as digitized clinical chart information of patients;
an input device inputting examination data, clinical finding data and treatment data as clinical chart information of a new patient;
an extraction device extracting clinical chart information of similar case patients similar to the input clinical chart information of the new patient from said database; and
a clinical chart information disclosing device disclosing to a viewer the contents of the extracted clinical chart information of similar case patients.

2. The electronic clinical chart system according to claim 1, wherein said extraction device compares examination data and clinical finding data of said new patient with examination data and clinical finding data of patients in past cases in said database to extract one or more data as data of similar case patients.

3. The electronic clinical chart system according to claim 2, wherein the similar case patient extracting device has a function of ordering according to the level of similarity using as an indicator the degree of similarity to examination data and clinical finding data of the new patient for one or more of extracted similar case patients.

4. The electronic clinical chart system according to claim 1, wherein the similar case patient clinical chart information disclosing device has a function of carrying out processing of making it impossible to view personal information which is not used in the diagnosis and treatment of similar case patients included in disclosed clinical chart information of the similar case patients, and is used only in identification of patients.

5. The electronic clinical chart system according to claim 1, wherein said patient electronic clinical chart database includes details of conducted examinations and the results of measurements as a part of a database of the results of examinations and measurements included in patients' examination data constituting the database.

6. The electronic clinical chart system according to claim 1, wherein the details of conducted examinations and the results of measurements included as a part of the database of the results of examinations and measurements included in patients' examination data include digitized data showing the results of probe hybridization reactions for a plurality of DNA probes using a DNA microarray.

7. The electronic clinical chart system according to claim 6, wherein digitized data showing the results of probe hybridization reactions for a plurality of DNA probes using said DNA microarray includes scan image data of label-originated signal intensities including spot regions of a plurality of DNA probes or label-originated signal intensity data at spots of a plurality of DNA probes when the results of probe hybridization reactions for a plurality of DNA probes are shown by label-originated signal intensities for identifying causal microorganism of an infectious disease.

8. The electronic clinical chart system according to claim 6, wherein the details of conducted examinations and the results of measurements included as a part of the database of the results of examinations and measurements included in patients' examination data include digitized data showing the results of probe hybridization reactions for a plurality of DNA probes for identifying causal microorganism of an infectious disease using the DNA microarray.


SEQUENCE LISTING (78 sequences)

SEQ ID NO: 1 - DNA, 23 bp, Artificial Sequence
  Forward primer F-1 for multiplex PCR amplification of coding region for 16s rRNA from genome DNA of bacteria
gcggcgtgcctaatacatgcaag

SEQ ID NO: 2 - DNA, 23 bp, Artificial Sequence
  Forward primer F-2 for multiplex PCR amplification of coding region for 16s rRNA from genome DNA of bacteria
gcggcaggcctaacacatgcaag

SEQ ID NO: 3 - DNA, 23 bp, Artificial Sequence
  Forward primer F-3 for multiplex PCR amplification of coding region for 16s rRNA from genome DNA of bacteria
gcggcaggcttaacacatgcaag

SEQ ID NO: 4 - DNA, 22 bp, Artificial Sequence
  Reverse primer R-1 for multiplex PCR amplification of coding region for 16s rRNA from genome DNA of bacteria
atccagccgcaccttccgatac

SEQ ID NO: 5 - DNA, 22 bp, Artificial Sequence
  Reverse primer R-2 for multiplex PCR amplification of coding region for 16s rRNA from genome DNA of bacteria
atccaaccgcaggttcccctac

SEQ ID NO: 6 - DNA, 22 bp, Artificial Sequence
  Reverse primer R-3 for multiplex PCR amplification of coding region for 16s rRNA from genome DNA of bacteria
atccagccgcaggttcccctac

SEQ ID NO: 7 - DNA, 26 bp, Artificial Sequence
  DNA probe A-1 for probe-hybridizat ion detection of coding region for 16s rRNA from genome DNA of Staphylococus aureus
gaaccgcatggttcaaaagtgaaaga

SEQ ID NO: 8 - DNA, 24 bp, Artificial Sequence
  DNA probe A-2 for probe-hybridization detection of coding region for 16s rRNA from genome DNA of Staphylococus aureus
cacttatagatggatccgcgctgc

SEQ ID NO: 9 - DNA, 26 bp, Artificial Sequence
  DNA probe A-3 for probe-hybridization detection of coding region for 16s rRNA from genome DNA of Staphylococus aureus
tgcacatcttgacggtacctaatcag

SEQ ID NO: 10 - DNA, 22 bp, Artificial Sequence
  DNA probe A-4 for probe-hybridization detection of coding region for 16s rRNA from genome DNA of Staphylococus aureus
ccccttagtgctgcagctaacg

SEQ ID NO: 11 - DNA, 23 bp, Artificial Sequence
  DNA probe A-5 for probe-hybridization detection of coding region for 16s rRNA from genome DNA of Staphylococus aureus
aatacaaagggcagcgaaaccgc

SEQ ID NO: 12 - DNA, 25 bp, Artificial Sequence
  DNA probe A-6 for probe-hybridization detection of coding region for 16s rRNA from genome DNA of Staphylococus aureus
ccggtggagtaaccttttaggagct

SEQ ID NO: 13 - DNA, 25 bp, Artificial Sequence
  DNA probe A-7 for probe-hybridization detection of coding region for 16s rRNA from genome DNA of Staphylococus aureus
taaccttttaggagctagccgtcga

SEQ ID NO: 14 - DNA, 23 bp, Artificial Sequence
  DNA probe A-8 for probe-hybridization detection of coding region for 16s rRNA from genome DNA of Staphylococus aureus
tttaggagctagccgtcgaaggt

SEQ ID NO: 15 - DNA, 23 bp, Artificial Sequence
  DNA probe A-9 for probe-hybridization detection of coding region for 16s rRNA from genome DNA of Staphylococus aureus
tagccgtcgaaggtgggacaaat

SEQ ID NO: 16 - DNA, 22 bp, Artificial Sequence
  DNA probe B-1 for probe-hybridization detection of coding region for 16s rRNA from genome DNA of Staphylococus
gaacagacgaggagcttgctcc

SEQ ID NO: 17 - DNA, 26 bp, Artificial Sequence
  DNA probe B-2 for probe-hybridization detection of coding region for 16s rRNA from genome DNA of Staphylococus
tagtgaaagacggttttgctgtcact

SEQ ID NO: 18 - DNA, 26 bp, Artificial Sequence
  DNA probe B-3 for probe-hybridization detection of coding region for 16s rRNA from genome DNA of Staphylococus
taagtaactatgcacgtcttgacggt

SEQ ID NO: 19 - DNA, 26 bp, Artificial Sequence
  DNA probe B-4 for probe-hybridization detection of coding region for 16s rRNA from genome DNA of Staphylococus
gacccctctagagatagagttttccc

SEQ ID NO: 20 - DNA, 24 bp, Artificial Sequence
  DNA probe B-5 for probe-hybridization detection of coding region for 16s rRNA from genome DNA of Staphylococus
agtaaccatttggagctagccgtc

SEQ ID NO: 21 - DNA, 23 bp, Artificial Sequence
  DNA probe B-6 for probe-hybridization detection of coding region for 16s rRNA from genome DNA of Staphylococus
gagcttgctcctctgacgttagc

SEQ ID NO: 22 - DNA, 22 bp, Artificial Sequence
  DNA probe B-7 for probe-hybridization detection of coding region for 16s rRNA from genome DNA of Staphylococus
agccggtggagtaaccatttgg

SEQ ID NO: 23 - DNA, 22 bp, Artificial Sequence
  DNA probe C-1 for probe-hybridization detection of coding region for 16s rRNA from genome DNA of Escherichia coli
ctcttgccatcggatgtgccca

SEQ ID NO: 24 - DNA, 26 bp, Artificial Sequence
  DNA probe C-2 for probe-hybridization detection of coding region for 16s rRNA from genome DNA of Escherichia coli
atacctttgctcattgacgttacccg

SEQ ID NO: 25 - DNA, 24 bp, Artificial Sequence
  DNA probe C-3 for probe-hybridization detection of coding region for 16s rRNA from genome DNA of Escherichia coli
tttgctcattgacgttacccgcag

SEQ ID NO: 26 - DNA, 24 bp, Artificial Sequence
  DNA probe C-4 for probe-hybridization detection of coding region for 16s rRNA from genome DNA of Escherichia coli
actggcaagcttgagtctcgtaga

SEQ ID NO: 27 - DNA, 23 bp, Artificial Sequence
  DNA probe C-5 for probe-hybridization detection of coding region for 16s rRNA from genome DNA of Escherichia coli
atacaaagagaagcgacctcgcg

SEQ ID NO: 28 - DNA, 25 bp, Artificial Sequence
  DNA probe C-6 for probe-hybridization detection of coding region for 16s rRNA from genome DNA of Escherichia coli
cggacctcataaagtgcgtcgtagt

SEQ ID NO: 29 - DNA, 26 bp, Artificial Sequence
  DNA probe C-7 for probe-hybridization detection of coding region for 16s rRNA from genome DNA of Escherichia coli
gcggggaggaagggagtaaagttaat

SEQ ID NO: 30 - DNA, 23 bp, Artificial Sequence
  DNA probe D-1 for probe-hybridization detection of coding region for 16s rRNA from genome DNA of Klebsiella
tagcacagagagcttgctctcgg

SEQ ID NO: 31 - DNA, 23 bp, Artificial Sequence
  DNA probe D-2 for probe-hybridization detection of coding region for 16s rRNA from genome DNA of Klebsiella
tcatgccatcagatgtgcccaga

SEQ ID NO: 32 - DNA, 25 bp, Artificial Sequence
  DNA probe D-3 for probe-hybridization detection of coding region for 16s rRNA from genome DNA of Klebsiella
cggggaggaaggcgataaggttaat

SEQ ID NO: 33 - DNA, 25 bp, Artificial Sequence
  DNA probe D-4 for probe-hybridization detection of coding region for 16s rRNA from genome DNA of Klebsiella
ttcgattgacgttacccgcagaaga

SEQ ID NO: 34 - DNA, 26 bp, Artificial Sequence
  DNA probe D-5 for probe-hybridization detection of coding region for 16s rRNA from genome DNA of Klebsiella
ggtctgtcaagtcggatgtgaaatcc

SEQ ID NO: 35 - DNA, 24 bp, Artificial Sequence
  DNA probe D-6 for probe-hybridization detection of coding region for 16s rRNA from genome DNA of Klebsiella
gcaggctagagtcttgtagagggg

SEQ ID NO: 36 - DNA, 24 bp, Artificial Sequence
  DNA probe E-1 for probe-hybridization detection of coding region for 16s rRNA from genome DNA of Pseudomonas aeruginosa
tgagggagaaagtgggggatcttc

SEQ ID NO: 37 - DNA, 25 bp, Artificial Sequence
  DNA probe E-2 for probe-hybridization detection of coding region for 16s rRNA from genome DNA of Pseudomonas aeruginosa
tcagatgagcctaggtcggattagc

SEQ ID NO: 38 - DNA, 24 bp, Artificial Sequence
  DNA probe E-3 for probe-hybridization detection of coding region for 16s rRNA from genome DNA of Pseudomonas aeruginosa
gagctagagtacggtagagggtgg

SEQ ID NO: 39 - DNA, 25 bp, Artificial Sequence
  DNA probe E-4 for probe-hybridization detection of coding region for 16s rRNA from genome DNA of Pseudomonas aeruginosa
gtacggtagagggtggtggaatttc

SEQ ID NO: 40 - DNA, 24 bp, Artificial Sequence
  DNA probe E-5 for probe-hybridization detection of coding region for 16s rRNA from genome DNA of Pseudomonas aeruginosa
gaccacctggactgatactgacac

SEQ ID NO: 41 - DNA, 25 bp, Artificial Sequence
  DNA probe E-6 for probe-hybridization detection of coding region for 16s rRNA from genome DNA of Pseudomonas aeruginosa
tggccttgacatgctgagaactttc

SEQ ID NO: 42 - DNA, 23 bp, Artificial Sequence
  DNA probe E-7 for probe-hybridization detection of coding region for 16s rRNA from genome DNA of Pseudomonas aeruginosa
ttagttaccagcacctcgggtgg

SEQ ID NO: 43 - DNA, 22 bp, Artificial Sequence
  DNA probe E-8 for probe-hybridization detection of coding region for 16s rRNA from genome DNA of Pseudomonas aeruginosa
tagtctaaccgcaagggggacg

SEQ ID NO: 44 - DNA, 22 bp, Artificial Sequence
  DNA probe F-1 for probe-hybridization detection of coding region for 16s rRNA from genome DNA of Serratia
tagcacagggagcttgctccct

SEQ ID NO: 45 - DNA, 25 bp, Artificial Sequence
  DNA probe F-2 for probe-hybridization detection of coding region for 16s rRNA from genome DNA of Serratia
aggtggtgagcttaatacgctcatc

SEQ ID NO: 46 - DNA, 26 bp, Artificial Sequence
  DNA probe F-3 for probe-hybridization detection of coding region for 16s rRNA from genome DNA of Serratia
tcatcaattgacgttactcgcagaag

SEQ ID NO: 47 - DNA, 26 bp, Artificial Sequence
  DNA probe F-4 for probe-hybridization detection of coding region for 16s rRNA from genome DNA of Serratia
actgcatttgaaactggcaagctaga

SEQ ID NO: 48 - DNA, 24 bp, Artificial Sequence
  DNA probe F-5 for probe-hybridization detection of coding region for 16s rRNA from genome DNA of Serratia
ttatcctttgttgcagcttcggcc

SEQ ID NO: 49 - DNA, 23 bp, Artificial Sequence
  DNA probe F-6 for probe-hybridization detection of coding region for 16s rRNA from genome DNA of Serratia
actttcagcgaggaggaaggtgg

SEQ ID NO: 50 - DNA, 25 bp, Artificial Sequence
  DNA probe G-1 for probe-hybridization detection of coding region for 16s rRNA from genome DNA of Streptococcus pneumoniae
agtagaacgctgaaggaggagcttg

SEQ ID NO: 51 - DNA, 25 bp, Artificial Sequence
  DNA probe G-2 for probe-hybridization detection of coding region for 16s rRNA from genome DNA of Streptococcus pneumoniae
cttgcatcactaccagatggacctg

SEQ ID NO: 52 - DNA, 26 bp, Artificial Sequence
  DNA probe G-3 for probe-hybridization detection of coding region for 16s rRNA from genome DNA of Streptococcus pneumoniae
tgagagtggaaagttcacactgtgac

SEQ ID NO: 53 - DNA, 26 bp, Artificial Sequence
  DNA probe G-4 for probe-hybridization detection of coding region for 16s rRNA from genome DNA of Streptococcus pneumoniae
gctgtggcttaaccatagtaggcttt

SEQ ID NO: 54 - DNA, 23 bp, Artificial Sequence
  DNA probe G-5 for probe-hybridization detection of coding region for 16s rRNA from genome DNA of Streptococcus pneumoniae
aagcggctctctggcttgtaact

SEQ ID NO: 55 - DNA, 24 bp, Artificial Sequence
  DNA probe G-6 for probe-hybridization detection of coding region for 16s rRNA from genome DNA of Streptococcus pneumoniae
tagaccctttccggggtttagtgc

SEQ ID NO: 56 - DNA, 26 bp, Artificial Sequence
  DNA probe G-7 for probe-hybridization detection of coding region for 16s rRNA from genome DNA of Streptococcus pneumoniae
gacggcaagctaatctcttaaagcca

SEQ ID NO: 57 - DNA, 24 bp, Artificial Sequence
  DNA probe H-1 for probe-hybridization detection of coding region for 16s rRNA from genome DNA of Haemophilus
gcttgggaatctggcttatggagg

SEQ ID NO: 58 - DNA, 23 bp, Artificial Sequence
  DNA probe H-2 for probe-hybridization detection of coding region for 16s rRNA from genome DNA of Haemophilus
tgccataggatgagcccaagtgg

SEQ ID NO: 59 - DNA, 26 bp, Artificial Sequence
  DNA probe H-3 for probe-hybridization detection of coding region for 16s rRNA from genome DNA of Haemophilus
cttgggaatgtactgacgctcatgtg

SEQ ID NO: 60 - DNA, 23 bp, Artificial Sequence
  DNA probe H-4 for probe-hybridization detection of coding region for 16s rRNA from genome DNA of Haemophilus
ggattgggcttagagcttggtgc

SEQ ID NO: 61 - DNA, 22 bp, Artificial Sequence
  DNA probe H-5 for probe-hybridization detection of coding region for 16s rRNA from genome DNA of Haemophilus
tacagagggaagcgaagctgcg

SEQ ID NO: 62 - DNA, 26 bp, Artificial Sequence
  DNA probe H-6 for probe-hybridization detection of coding region for 16s rRNA from genome DNA of Haemophilus
ggcgtttaccacggtatgattcatga

SEQ ID NO: 63 - DNA, 23 bp, Artificial Sequence
  DNA probe H-7 for probe-hybridization detection of coding region for 16s rRNA from genome DNA of Haemophilus
aatgcctaccaagcctgcgatct

SEQ ID NO: 64 - DNA, 25 bp, Artificial Sequence
  DNA probe H-8 for probe-hybridization detection of coding region for 16s rRNA from genome DNA of Haemophilus
tatcggaagatgaaagtgcgggact

SEQ ID NO: 65 - DNA, 22 bp, Artificial Sequence
  DNA probe I-1 for probe-hybridization detection of coding region for 16s rRNA from genome DNA of Enterobac yer cloacae
cagagagcttgctctcgggtga

SEQ ID NO: 66 - DNA, 26 bp, Artificial Sequence
  DNA probe I-2 for probe-hybridization detection of coding region for 16s rRNA from genome DNA of Enterobacyer cloacae
gggaggaaggtgttgtggttaataac

SEQ ID NO: 67 - DNA, 26 bp, Artificial Sequence
  DNA probe I-3 for probe-hybridization detection of coding region for 16s rRNA from genome DNA of Enterobacyer cloacae
ggtgttgtggttaataaccacagcaa

SEQ ID NO: 68 - DNA, 22 bp, Artificial Sequence
  DNA probe I-4 for probe-hybridization detection of coding region for 16s rRNA from genome DNA of Enterobacyer cloacae
gcggtctgtcaagtcggatgtg

SEQ ID NO: 69 - DNA, 25 bp, Artificial Sequence
  DNA probe I-5 for probe-hybridization detection of coding region for 16s rRNA from genome DNA of Enterobacyer cloacae
attcgaaactggcaggctagagtct

SEQ ID NO: 70 - DNA, 25 bp, Artificial Sequence
  DNA probe I-6 for probe-hybridization detection of coding region for 16s rRNA from genome DNA of Enterobacyer cloacae
taaccacagcaattgacgttacccg

SEQ ID NO: 71 - DNA, 24 bp, Artificial Sequence
  DNA probe I-7 for probe-hybridization detection of coding region for 16s rRNA from genome DNA of Enterobacyer cloacae
gcaattgacgttacccgcagaaga

SEQ ID NO: 72 - DNA, 23 bp, Artificial Sequence
  DNA probe J-1 for probe-hybridization detection of coding region for 16s rRNA from genome DNA of Enterococcus fecaris
ttctttcctcccgagtgcttgca

SEQ ID NO: 73 - DNA, 25 bp, Artificial Sequence
  DNA probe J-2 for probe-hybridization detection of coding region for 16s rRNA from genome DNA of Enterococcus fecaris
aacacgtgggtaacctacccatcag

SEQ ID NO: 74 - DNA, 24 bp, Artificial Sequence
  DNA probe J-3 for probe-hybridization detection of coding region for 16s rRNA from genome DNA of Enterococcus fecaris
atggcataagagtgaaaggcgctt

SEQ ID NO: 75 - DNA, 22 bp, Artificial Sequence
  DNA probe J-4 for probe-hybridization detection of coding region for 16s rRNA from genome DNA of Enterococcus fecaris
gacccgcggtgcattagctagt

SEQ ID NO: 76 - DNA, 25 bp, Artificial Sequence
  DNA probe J-5 for probe-hybridization detection of coding region for 16s rRNA from genome DNA of Enterococcus fecaris
ggacgttagtaactgaacgtcccct

SEQ ID NO: 77 - DNA, 22 bp, Artificial Sequence
  DNA probe J-6 for probe-hybridization detection of coding region for 16s rRNA from genome DNA of Enterococcus fecaris
ctcaaccggggagggtcattgg

SEQ ID NO: 78 - DNA, 22 bp, Artificial Sequence
  DNA probe J-7 for probe-hybridization detection of coding region for 16s rRNA from genome DNA of Enterococcus fecaris
ttggagggtttccgcccttcag